(19) United States
(12) Patent Application Publication
(10) Pub. No.: US 2026/0152536 A1
(43) Pub. Date: Jun. 4, 2026

(54) CHIMERIC PROTEINS COMPRISING MEMBRANE BOUND IL-12 WITH PROTEASE CLEAVABLE LINKERS

(71) Applicant: Regeneron Pharmaceuticals, Inc., Tarrytown, NY (US)

(72) Inventor: Saida DADI-MEHMETAJ, Tarrytown, NY (US)

(73) Assignee: Regeneron Pharmaceuticals, Inc., Tarrytown, NY (US)

(21) Appl. No.: 19/122,945

(22) PCT Filed: Nov. 14, 2023
(86) PCT No.: PCT/US2023/079557

§ 371 Date: Apr. 21, 2025

Related U.S. Application Data
(60) Provisional application No. 63/383,943, filed on Nov. 16, 2022.

(51) Int. Cl. C07K 14/54; A61K 9/00; A61K 31/7105; A61K 35/12; A61K 38/00; A61P 35/00; C12N 5/071; C12N 9/50; C12N 15/62; C12N 15/86

(52) U.S. Cl. CPC C07K 14/5434; A61K 9/0019; A61K 31/7105; A61K 35/12; A61K 38/00; A61P 35/00; C12N 5/0602; C12N 9/50; C12N 15/625; C12N 15/86; C07K 2319/02; C07K 2319/03; C12N 2830/002; C12N 2830/008; C12N 2830/34

(57) ABSTRACT

The present disclosure provides chimeric proteins that are designed to target cell-based immunotherapy more specifically to tumors and the tumor microenvironment. Use of membrane-bound effector molecules reduces the off-target side effects and toxicity of the immunotherapy. The chimeric protein of the disclosure comprises an effector molecule, a transmembrane domain, and a protease cleavable linker. The present disclosure also provides nucleic acid constructs expressing the chimeric protein of the present disclosure. Pharmaceutical compositions comprising the chimeric proteins of the present disclosure are also provided.

### CROSS REFERENCE TO RELATED APPLICATIONS

The present application claims priority to U.S. Provisional Application No. 63/383,943, filed 16 Nov. 2022, which is hereby incorporated in its entirety.

### SEQUENCE LISTING

The instant application contains a Sequence Listing which has been submitted electronically in XML format and is hereby incorporated by reference in its entirety. Said XML copy, created on Oct. 19, 2023, is named 67000-1381_WO_SL.xml and is 35,788 bytes in size.

### FIELD OF THE INVENTION

The present invention is in the field of targeted cell based therapy to treat diseases. The present invention relates to the preparation and use of treatments based on membrane-cleavable chimeric proteins.

### BACKGROUND OF THE INVENTION

Conventional therapies for cancer treatment, such as chemotherapy and radiation treatment, have limited efficacy and are often associated with systemic toxicity, and off-target adverse events. Cell-based therapy is designed to localize treatment to the tumor environment, avoiding off target adverse effects. One such cell-based therapy is chimeric antigen receptor (CAR) T-cell therapy. In CAR T-cell therapy, T-cells are engineered to express a synthetic receptor, which redirects effector function to the tumor or tumor microenvironment (TME).

### SUMMARY OF THE INVENTION

The present invention provides chimeric proteins that are designed to target cell-based immunotherapy more specifically to tumors and the TME. Use of membrane-bound effector molecules reduces the off-target side effects and toxicity of the immunotherapy. The chimeric proteins of the present invention are suitable for use in armored CAR T-cell therapy.

In a particular aspect, the present invention provides a membrane-cleavable chimeric protein, oriented from N-terminal to C-terminal, having the formula:

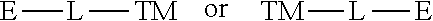

wherein:
a) E comprises an effector molecule,
b) L is a first peptide linker comprising a protease cleavage site and an amino acid sequence selected from the group consisting of: GGGGSISSGLLSGRSDNHGGGGS (SEQ ID NO: 3); GGGGSVPLSLYSGGGISSGLLSGRSDNHGGGGS (SEQ ID NO: 4); GGGGSHPVGLLARGGGHPVGLLARGGSGRSAGGSGRSAGGGGS (SEQ ID NO: 5); GGGGSHPVGLLARGGGGS (SEQ ID NO: 6); and GGGGSLAQAVRSSGGGGS (SEQ ID NO: 7),
c) TM comprises a cell transmembrane protein, and
d) E-L-TM or TM-L-E is configured to be expressed as a single polypeptide.

In certain embodiments, the effector molecule E comprises a cytokine or a functional fragment thereof. The cytokine is selected from the group consisting of IL-1-beta, IL-2, IL-4, IL-6, IL-7, IL-10, IL-12, IL-15, IL-17A, IL-18, IL-21, IL-22, Type I interferons, interferon-gamma, and tumor necrosis factor-alpha (TNF-alpha). In some embodiments, the cytokine is IL-12. In some embodiments, the effector molecule E comprises the p40 and p35 subunits of IL-12, covalently linked by a second peptide linker. An IL-12 molecule that comprises both the s p40 and p35 subunits is often referred to as IL-12p70 or IL-12p70 fusion protein.

In some embodiments, the transmembrane domain is derived from the group consisting of PDGFR-beta, CD8, CD28, CD3 zeta-chain, CD4, 4-1BB, OX40, ICOS, CTLA-4, PD-1, LAG03, 2B4, LNGFR, NKG2D, TNFR2, B7-1, and BTLA. In some embodiments, the transmembrane is linked to a cytoplasmic domain. In certain embodiments, the cytoplasmic domain is derived from PDGFR-beta, CD8, CD28, CD3 zeta-chain, CD4, 4-1BB, 0X40, ICOS, CTLA-4, PD-1, LAG-3, 2B4, LNGFR, NKG2D, EpoR, TNFR2, B7-1, or BTLA.

In some embodiments, the chimeric protein comprises a signal peptide at the N-terminus.

In addition to the disclosed peptide sequences, the present disclosure also includes nucleic acids (mRNA, DNA, siRNA, etc.) that express all or a portion of the peptide sequences. In a particular aspect, the present disclosure provides a nucleic acid molecule coding for the chimeric protein of the disclosure. In some embodiments, the nucleic acid molecule comprises an expression cassette, wherein the expression cassette comprises a promoter and an exogenous polynucleotide sequence encoding the chimeric protein of the present disclosure.

In certain embodiments, the present disclosure provides an expression vector comprising the nucleic acid molecule that codes for the chimeric protein of the disclosure. In some embodiments, the expression vector is a viral vector.

In some aspects, the present disclosure provides a cell that comprises the chimeric protein of the disclosure, the nucleic acid encoding the chimeric protein of the disclosure, or the expression vector that comprises the chimeric protein of the disclosure. In some embodiments, the cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell. In some embodiments the cell further comprises a protease capable of cleaving the protease cleavage site L. Advantageously the protease is an endogenous protease.

In other aspects, the present disclosure provides a pharmaceutical composition comprising the chimeric protein of the disclosure, the nucleic acid encoding the chimeric protein of the disclosure, the expression vector that comprises the chimeric protein of the disclosure, or the isolated cell of the disclosure, and a pharmaceutically acceptable excipient. In preferred embodiments the pharmaceutical composition is a lipid nanoparticle (LNP) pharmaceutical composition.

The present disclosure also provides a method of treating a disease in a subject in need thereof, the method comprising administering a therapeutically effective amount of the pharmaceutical composition of the disclosure. In certain embodiments, the disease is cancer.

### BRIEF DESCRIPTION OF THE DRAWINGS

FIG. 1: FIG. 1 is a schematic illustrating the tested protease-cleavable linker coding sequences. IL12p40 linked to IL12p35 forms the bioactive IL-12p70 protein fused to various non-cleavable (NC, L1) or cleavable (L2-L5) substrates that is fused to transmembrane domain of B7.1. In the presence of proteases, binding to and enzymatic cleavage of their respective substrate sites will release IL-12p70 from being tethered to the transmembrane.

FIGS. 2A, 2B and 2C: FIGS. 2A, 2 B and 2C illustrate cell surface staining of CHO cells that were transfected overnight with 500 ng of mRNA constructs encoding for F-luciferase, soluble IL-12p70, transmembrane anchored IL-12p70 (TM_IL12, L1) or various transmembrane anchored IL-12p70 constructs harboring protease-cleavable domains (L2, L3, L4, L4short, L5). Cells were then treated with PBS or activated murine MMP2 (1 μg/mL) for 180 minutes at which point cells were harvested for flow cytometric staining of surface IL12p40.

FIG. 3: FIG. 3 depicts the concentration of soluble IL-12p70 released from CHO cells that were transfected overnight with 500 ng of mRNA constructs encoding for soluble IL-12p70, transmembrane anchored IL-12p70 (TM_IL12, L1) or various transmembrane anchored IL-12p70 constructs harboring protease-cleavable domains (L2, L3, L4, L4short, L5). Cells were then treated with activated murine MMP2 (lug/mL) for 180 minutes and the supernatant was collected for detection of cleaved/soluble IL-12p70 by ELISA.

FIG. 4: FIG. 4 illustrates tumor growth over time, measured as tumor volume, in animals bearing B16F10 tumors. The animals were randomized to treatment groups when the tumors reached a size of 100 mm³ and they were treated intratumorally on Days 0, 3, 6 and 9 with 10 μg of one of the mRNA encoding for soluble (dashed), membrane-bound (dotted) or protease-cleavable (solid) membrane bound IL-12p70. Average tumor growth over time was quantified.

FIG. 5: FIG. 5 is a bar graph illustrating the average tumor volume in animals bearing B16F10 tumors that were randomized at 100 mm³ and treated on Days 0, 3, 6 and 9 with 10 μg of each mRNA encoding for soluble, membrane-bound or protease-cleavable membrane bound IL-12p70. FIG. 5 is a representative bar graph of average tumor sizes at day 11 post-treatment before control F-luciferase-treated animals had to be euthanized.

FIG. 6: FIG. 6 shows Kaplan-Meier survival curves of B16F10 tumor-bearing animals treated intratumorally on Days 0, 3, 6 and 9 with 10 μg of each mRNA encoding for soluble (dashed), membrane-bound (dotted) or protease-cleavable membrane bound (solid) IL-12p70. Animals were euthanized when tumors reached sizes >2000 mm³.

FIG. 7: FIG. 7 shows levels of circulating IL-12p70 from B16F10 tumor-bearing animals (N=5/group) that were bled 24 hours post-treatment with 10 μg of each LNP-mRNA encoding for soluble, transmembrane-bound or protease-cleavable IL-12p70. Serum was subjected to ELISA to quantify circulating IL-12p70 levels.

FIG. 8: FIG. 8 illustrates average tumor growth over time, as tumor volume, in animals bearing MC38 tumors that were randomized at 85 mm³ and treated on Days 0, 3, 6 and 9 with 10 μg of each mRNA encoding for soluble (dashed), membrane-bound (dotted) or protease-cleavable membrane bound (solid) IL-12p70. Average tumor growth over time was quantified.

FIG. 9: FIG. 9 illustrates the average tumor volume in animals bearing MC38 tumors that were randomized at 85 mm³ and treated on Days 0, 3, 6 and 9 with 10 μg of each mRNA encoding for soluble, membrane-bound or protease-cleavable membrane bound IL-12p70. Representative bar graph of average tumor sizes at day 13 post-treatment before control F-luciferase-treated animals had to be euthanized.

FIG. 10: FIG. 10 shows Kaplan-Meier survival curves of MC38 tumor-bearing animals treated intratumorally on Days 0, 3, 6 and 9 with 10 ug of each mRNA encoding for soluble (dashed), membrane-bound (dotted) or protease-cleavable membrane bound (solid) IL-12p70. Animals were euthanized when tumors reached sizes >2000 mm³.

FIG. 11: FIG. 11 illustrates levels of circulating IL-12p70 in MC38 tumor-bearing animals (N=5/group) that were bled 24 hours post-treatment with 10 μg of each LNP-mRNA encoding for soluble, membrane-bound or protease-cleavable membrane bound IL-12p70. Serum was subjected to serum ELISA to quantify circulating IL-12p70 levels.

### DETAILED DESCRIPTION OF THE INVENTION

Cell-based therapies are of interest for the treatment of various diseases because of the possibility of reducing off-target adverse effects. One such cell-based therapy is chimeric antigen receptor (CAR) T-cell therapy. T-cells are engineered to express a synthetic receptor, which redirects effector function to the tumor or tumor microenvironment (TME). Although this approach has been effective in treating hematological malignancies, treatment of solid tumors has not been as successful. The immunosuppressive solid tumor TME is a significant barrier, and reduces the anti-cancer activity of endogenous tumor-resident immune cells, which allows for tumor growth. Recently, there have been efforts to enhance CAR T-cell function in the TME by engineering the cells to express other proteins alongside the CAR. Examples of this engineering include inducing CAR T-cells to secrete cytokines or express cytokine receptors to modulate cytokine activity in the TME, or producing CAR T-cells that secrete antibody-like proteins that target tumor antigens. These methods are known as “armored CAR T-cell therapy.”

Interleukins have been extensively studied in the treatment of cancer. However, interleukins may have both cancer exacerbation effects, as well as cancer inhibition effects. In addition, administration of interleukins is often associated with adverse effects, including lethality. For example, CAR T-cells engineered to express interleukins have been shown to induce “cytokine release syndrome,” leading to febrile neutropenia, hypotension, acute vascular leakage syndrome, and acute respiratory distress syndrome. Therefore, treatment with interleukins has proven to be challenging. Efforts to specifically target interleukins to cancer cells are in progress.

The present disclosure provides chimeric proteins that are designed to target cell-based immunotherapy more specifically to tumors and the TME. Use of membrane-bound effector molecules reduces the off-target side effects and toxicity of the immunotherapy.

The present disclosure also provides nucleic acids expressing the chimeric proteins of the present disclosure.

In some embodiments, the present disclosure provides expression vectors comprising either the chimeric proteins of the disclosure, or nucleic acids that express the chimeric proteins of the disclosure. Expression vectors include viral vectors.

Any host-vector system known to those of skill in the art can be used to express the protein coding sequence. For example, a mammalian cell system infected with a virus vector comprising the nucleic acid sequence can be used. Any method known to one of skill in the art can be used to insert DNA fragments into a vector, and to construct expression vectors containing a chimeric gene containing appropriate transcriptional/translational control signals and protein coding sequences.

The chimeric proteins, nucleic acids, or expression vectors may be provided as a pharmaceutical composition. In preferred embodiments, the pharmaceutical composition is provided in a lipid nanoparticle.

It is to be understood that the foregoing general description and the following detailed description are exemplary and explanatory only, and are not restrictive of any subject matter claimed.

Headings are used solely for organizational purposes, and are not intended to limit the disclosure in any way.

Unless otherwise defined, all technical and scientific terms used herein have the same meaning as is commonly understood by one of skill in the art to which the disclosures belong. All patents, patent applications, published applications and publications, websites and other published materials referred to throughout the entire disclosure herein, unless noted otherwise, are incorporated by reference in their entirety for any purpose. Although any methods and materials similar or equivalent to those described herein can be used in the practice or testing of the present disclosure, the preferred methods are described.

### Definitions

In this application, the use of the singular includes the plural unless specifically stated otherwise. As used herein, the singular forms “a,” “an” and “the” are intended to include the plural forms as well, unless the context clearly indicates otherwise.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Unless otherwise indicated, statistically significant means p<0.05.

As used herein, “treatment” refers to any delivery, administration, or application of a therapeutic for a disease or condition. Treatment may include curing the disease, inhibiting the disease, slowing or stopping the development of the disease, ameliorating one or more symptoms of the disease, or preventing the recurrence of one or more symptoms of the disease

In this application, the use of “or” means “and/or” unless stated otherwise. Also, when it is clear from the context in which it is used, “and” may be interpreted as “or,” such as in a list of alternatives where it is not possible for all to be true or present at once.

As used herein, the terms “comprises” and/or “comprising” specify the presence of the stated features, integers, steps, operations, elements, and/or components, but do not preclude the presence or addition of one or more other features, integers, steps, operations, elements, components, and/or groups thereof that are not specifically recited. For example, a composition that “comprises” or “includes” a protein may contain the protein alone or in combination with other ingredients. Furthermore, to the extent that the terms “includes,” “having,” “has,” “with,” “composed,” “comprised” or variants thereof are used in either the detailed description or the claims, such terms are intended to be inclusive in a manner similar to the term “comprising.”

The transitional phrase “consisting essentially of” means that the scope of a claim is to be interpreted to encompass the specified elements recited in the claim and those that do not materially affect the basic and novel characteristic(s) of the claimed disclosure. Thus, the term “consisting essentially of” when used in a claim of this disclosure is not intended to be interpreted to be equivalent to “comprising.”

When the terms “consist of”, “consists of” or “consisting of” is used in the body of a claim, the claim term set off with “consist of”, “consists of” and/or “consisting of” is limited to the elements recited immediately following “consist of”, “consists of” and/or “consisting of”, and is closed to un-recited elements related to that particular claim term. The term ‘combinations thereof’, when included in the listing of the recited elements that follow “consist of”, “consists of” and/or “consisting of” means a combination of only two or more of the elements recited.

“Optional” or “optionally” means that the subsequently described event or circumstance may or may not occur and that the description includes instances in which the event or circumstance occurs and instances in which the event or circumstance does not.

As used herein, ranges and amounts can be expressed as “about” a particular value or range. “About” is intended to also include the exact amount. Hence “about 5 percent” means “about 5 percent” and also “5 percent.” “About” means within typical experimental error for the application or purpose intended. Unless otherwise apparent from the context, the term “about” encompasses values within a standard margin of error of measurement (e.g., SEM) of a stated value.

It is to be understood that wherein a numerical range is recited, it includes the end points, all values within that range, and all narrower ranges within that range, whether specifically recited or not.

As used herein, “effector molecule” is a molecule (e.g. ligand) that binds to another molecule, e.g. a receptor, and modulates the biological activity of the molecule to which it binds. An effector molecule may, for example, modulate enzymatic activity, gene expression, or cell signaling. An effector molecule may be a peptide, a protein (polypeptide), or a nucleic acid such as DNA or RNA. Effector molecules that modulate immune response are of particular for the present disclosure.

As used herein, “transmembrane protein” means a protein that spans the distance through a cell wall lipid bilayer. Transmembrane proteins have 3 regions. One region is outside the cell, and is called the “extracellular domain.” Another region is within the lipid bilayer of the cell wall, is called the “transmembrane domain,” and is generally referred as “TM” for the purposes of the present disclosure. The third region is inside the cell, is called the “cytoplasmic domain,” and is generally referred to as “CD” for the purposes of the present disclosure. When the TM is linked to a CD in the chimeric protein of the present disclosure, the construct is referred to as a “TMCD.”

As used herein, “TME” refers to the tumor microenvironment.

The term “isolated” with respect to proteins, nucleic acids, and cells includes proteins, nucleic acids, and cells that are relatively purified with respect to other cellular or organism components that may normally be present in situ, up to and including a substantially pure preparation of the protein, nucleic acid, or cell. The term “isolated” also includes proteins and nucleic acids that have no naturally occurring counterpart or proteins or nucleic acids that have been chemically synthesized and are thus substantially uncontaminated by other proteins or nucleic acids. The term “isolated” also includes proteins, nucleic acids, or cells that have been separated or purified from most other cellular components or organism components with which they are naturally accompanied (e.g., other cellular proteins, nucleic acids, or cellular or extracellular components).

The term “variant” refers to a nucleotide sequence differing from the sequence most prevalent in a population (e.g., by one nucleotide) or a protein sequence different from the sequence most prevalent in a population (e.g., by one amino acid).

The term “fragment,” when referring to a protein, means a protein that is shorter or has fewer amino acids than the full-length protein. The term “fragment,” when referring to a nucleic acid, means a nucleic acid that is shorter or has fewer nucleotides than the full-length nucleic acid. A fragment can be, for example, when referring to a protein fragment, an N-terminal fragment (i.e., removal of a portion of the C-terminal end of the protein), a C-terminal fragment (i.e., removal of a portion of the N-terminal end of the protein), or an internal fragment (i.e., removal of a portion of each of the N-terminal and C-terminal ends of the protein). A fragment can be, for example, when referring to a nucleic acid fragment, a 5′ fragment (i.e., removal of a portion of the 3′ end of the nucleic acid), a 3′ fragment (i.e., removal of a portion of the 5′ end of the nucleic acid), or an internal fragment (i.e., removal of a portion each of the 5′ and 3′ ends of the nucleic acid).

“Sequence identity” or “identity” in the context of two polynucleotides or polypeptide sequences refers to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue.

A “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologous sequence and paralogous sequences. Homologous genes, for example, typically descend from a common ancestral DNA sequence, either through a speciation event (orthologous genes) or a genetic duplication event (paralogous genes). “Orthologous” genes include genes in different species that evolved from a common ancestral gene by speciation. Orthologs typically retain the same function in the course of evolution. “Paralogous” genes include genes related by duplication within a genome. Paralogs can evolve new functions in the course of evolution.

The term “in vitro” includes artificial environments and to processes or reactions that occur within an artificial environment (e.g., a test tube or in isolated cell or cell line). The term “in vivo” includes natural environments (e.g., a cell or organism or body) and to processes or reactions that occur within a natural environment. The term “ex vivo” includes cells that have been removed from the body of an individual and processes or reactions that occur within such cells.

The term “reporter gene” refers to a nucleic acid having a sequence encoding a gene product (typically an enzyme) that is easily and quantifiably assayed when a construct comprising the reporter gene sequence operably linked to an endogenous or heterologous promoter and/or enhancer element is introduced into cells containing (or which can be made to contain) the factors necessary for the activation of the promoter and/or enhancer elements. Examples of reporter genes include, but are not limited, to genes encoding beta-galactosidase (lacZ), the bacterial chloramphenicol acetyltransferase (cat) genes, firefly luciferase genes, genes encoding beta-glucuronidase (GUS), and genes encoding fluorescent proteins. A “reporter protein” refers to a protein encoded by a reporter gene.

The term “fluorescent reporter protein” as used herein means a reporter protein that is detectable based on fluorescence wherein the fluorescence may be either from the reporter protein directly, activity of the reporter protein on a fluorogenic substrate, or a protein with affinity for binding to a fluorescent tagged compound. Examples of fluorescent proteins include green fluorescent proteins (e.g., GFP, GFP-2, tagGFP, turboGFP, eGFP, Emerald, Azami Green, Monomeric Azami Green, CopGFP, AceGFP, and ZsGreen1), yellow fluorescent proteins (e.g., YFP, eYFP, Citrine, Venus, YPet, PhiYFP, and ZsYellow1), blue fluorescent proteins (e.g., BFP, eBFP, eBFP2, Azurite, mKalamal, GFPuv, Sapphire, and T-sapphire), cyan fluorescent proteins (e.g., CFP, eCFP, Cerulean, CyPet, AmCyanl, and Midoriishi-Cyan), red fluorescent proteins (e.g., RFP, mKate, mKate2, mPlum, DsRed monomer, mCherry, mRFP1, DsRed-Express, DsRed2, DsRed-Monomer, HcRed-Tandem, HcRed1, AsRed2, eqFP611, mRaspberry, mStrawberry, and Jred), orange fluorescent proteins (e.g., mOrange, mKO, Kusabira-Orange, Monomeric Kusabira-Orange, mTangerine, and tdTomato), and any other suitable fluorescent protein whose presence in cells can be detected by flow cytometry methods.

As used herein, “subject” means a living organism. Preferably, a subject is a mammal, such as a human, non-human primate, rodent, or companion animal such as a dog, cat, cow, pig, etc.

### Chimeric Proteins

The chimeric proteins of the present disclosure comprise an effector molecule, a protease cleavable linker, and a transmembrane protein, preferably the transmembrane domain and/or the cytoplasmic domain of the transmembrane protein. The chimeric proteins of the disclosure are suitable for treating various types of cancer.

The proteins of the disclosure are membrane-cleavable chimeric proteins, oriented from N-terminal to C-terminal, having the formula:

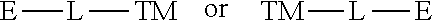

E is an effector molecule that is a moiety that elicits a biological effect, such as, for example, inhibiting the growth of cancerous cells, and/or inhibiting metastases or spreading of cancerous cells, and/or reducing the number of cancerous cells, and/or reducing the volume of pre-existing tumors. Preferably, the effector molecule is an immunomodulatory molecule, such as a cytokine or a portion thereof. Suitable effector molecules of the present disclosure are described in more detail below.

L is a first peptide linker. In some embodiments, L can comprise a protease cleavable peptide. Preferably, L is cleavable by a protease that is expressed in cancer cells. L links the effector molecule E to the transmembrane protein TM. L is described in more detail below.

TM is the transmembrane portion of a transmembrane protein that can be expressed in a cell used for cell-based therapy. In some embodiments, the TM is linked to a cytoplasmic domain of a transmembrane protein, and is referred to as TMCD.

In the tumor or the TME, a protease expressed on a cell (for example, a cell expressing the chimeric protein of the disclosure, or a cancer cell) cleaves the linker L, thereby releasing the effector molecule from the cell membrane. Upon release, the effector molecule elicits the desired biological effect on the cancer cells.

As used herein, “first linker” refers to the protease cleavable linker “L”.

As used herein, “second linker” refers to the peptide linker that covalently bonds the alpha and beta subunits of the IL-12 family of cytokines.

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

Proteins are said to have an “N-terminus” and a “C-terminus.” The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

### Effector Molecule

The effector molecule E of the present disclosure can comprise cytokines or functional parts thereof. Cytokines are a category of small proteins between about 5-20 kDa that are involved in cell signaling and include chemokines, interferons (INF), interleukins (IL), and tumor necrosis factors (TNF), among others. Chemokines play a role as a chemo-attractant to guide the migration of cells and are classified into four subfamilies: CXC, CC, CX3C, and XC. Exemplary chemokines include chemokines from the CC subfamily, such as CCL1, CCL2 (MCP-1), CCL3, CCL4, CCL5 (RANTES), CCL6, CCL7, CCL8, CCL9 (or CCL10), CCL11, CCL12, CCL13, CCL14, CCL15, CCL16, CCL17, CCL18, CCL19, CCL20, CCL21, CCL22, CCL23, CCL24, CCL25, CCL26, CCL27, and CCL28; the CXC subfamily, such as CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, and CXCL17; the XC subfamily, such as XCL1 and XCL2; and the CX3C subfamily, such as CX3CL1.

Effector molecules may be a co-stimulatory domain. In some cases, the co-stimulatory domain enhances antigen-specific cytotoxicity. In some cases, the co-stimulatory domain further enhances cytokine production. In some embodiments, the co-stimulatory domain comprises CD27, CD28, CD70, CD80, CD83, CD86, CD134 (OX-40), CD134L (OK-40L), CD137 (41BB), CD137L (41BBL), or CD224.

In some embodiments, the effector molecule is an immune checkpoint inhibitor polypeptide that inhibits a negative regulatory molecule of T-cell activation. Immune checkpoint inhibitor bind to immune checkpoint molecules, which are a group of molecules on the cell surface of CD4 and CD8 T cells. Exemplary immune checkpoint molecules include, but are not limited to, programmed death-ligand 1 (PDL1, also known as B7-H1, CD274), programmed death 1 (PD-1), PD-L2 (B7-DC, CD273), LAG3, TIM3, 2B4, A2aR, B7H1, 87H3, B7H4, BTLA, CD2, CD16, CD27, CD28, CD30, CD40, CD70, CD80, CD86, CD137, CD160, CD226, CD276, DR3, GAL9, GITR, HAVCR2, HVEM, IDO1, IDO2, inducible T cell costimulatory (ICOS), KIR, LAIR, LIGHT, macrophage receptor with collagencous structure (MARCO), OX-40, phosphatidylserine (PS), SLAM, TIGHT, VISTA, and VTCN1. In some embodiments, an immune checkpoint inhibitor inhibits on or more of PDL1, PD-1, CTLA-4, PD-L2, LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS, KIR, LAIR1, LIGHT, MARCO, OX-40, PS, SLAM, TIGHT, VISTA, and VTCN1.

Suitable cytokines to be used as effector molecules in the present disclosure include, but are not limited to, IL-1-beta, IL-2, IL-4, IL-6, IL-7, IL-10, IL-12, IL-15, IL-17A, IL-18, IL-21, IL-22, Type I interferons (e.g. IFN-alpha, IFN-beta), interferon-gamma (IFN-gamma), and tumor necrosis factor-alpha (TNF-alpha).

Cytokines are produced by several types of cells, including endothelial cells, fibroblasts, and stromal cells, as well as several immune cells such as macrophages, B lymphocytes, T lymphocytes, and mast cells. Their actions are mediated by cell surface receptors. Cytokine activity via immune cells is of interest for certain diseases, such as cancer. However, use of cytokines, such as interleukins, has been limited by their toxic effects, as mentioned above.

IFN-alpha immunocytokines have been demonstrated to exert an anti-tumor effect mediated by the activation of immune system cells. IFN-alpha has been used for the treatment of hematological malignancies and solid tumors. IFN-alpha has direct pro-apoptic/anti-proliferative activity on tumor cells. Of note is that high doses of IFN-alpha are antiangiogenic, thereby exerting anti-tumor activity by affecting tumor vasculature, and reducing blood flow to tumors. However, in subjects having cancer, systemic treatment with IFN-alpha is associated with toxicity. Attempts have been made to reduce toxicity by fusion to apolipoprotein A-1—the apolipoprotein A-1 moiety incorporates the cytokine into high-density lipoproteins, improving the pharmacokinetics and anti-tumor activity of the IFN-alpha. Another method by which toxicity can be attenuated is by using AcTakines (activity-on-Target cytokines). The AcTakines technology is based on the fusion of a mutated cytokine that shows reduced affinity for its receptor to a cell-specific targeting domain. IFN-alpha fused to single domain antibodies targeting Clec9A, which is a molecule expressed on dendritic cells (DCs) specialized in cross-priming, also displays a potent anti-tumor effect.

IL-15 is mainly produced by activated myeloid cells as a membrane-bound heterodimer associated with the receptor IL-15R-alpha is such a way that it is trans-represented to NK cells and T cells expressing IL-2/IL-15R-beta. IL-15 is critically needed for the ontogeny of NK cells and CD8+ T cells, and also induces the proliferation, cytotoxic action, and the release of other cytokines such as IFN-gamma, from these cells. IL-15 does not stimulate Treg cells.

IL-10 is released by innate and adaptive immune cells to fine-tune the activity of pro-inflammatory cytokines. IL-10 is considered to be an immunosuppressive cytokine as IL-10 can decrease the antigen-presenting activity of DCs, and inhibit the cytotoxic and cytokine-release functions performed by T and NK lymphocytes. IL-10 activity may also be context-dependent. For example, in chronic infections and cancer, autocrine IL-10 activity on CD8+ T lymphocytes can be crucial for inhibiting antigen-induced CD8+ T cell apoptosis, thereby prolonging the effector activity of these cytotoxic lymphocytes.

IL-2 can be a key cytokine in promoting the expansion of natural killer (NK) and T lymphocytes. Improvement of the pharmacokinetic profile of IL-2 can be achieved by covalent binding of IL-2 to moieties that increase the half-life in circulation (e.g. such as Fc domains of immunoglobulins or polyethylene glycol (PEG)), or by chimerization with antibodies that target the cytokine to the TME. Improvement of the pharmacodynamics properties of IL-2 can be achieved by using technologies that reduce binding to the high-affinity IL-2 receptor, while maintaining binding to the medium-affinity IL-2 receptor, to increase the amount of the cytokine that is available to stimulate NK and T cells.

IL-12 can have significant anti-tumor activity by triggering anti-tumor immune responses. However, administration of IL-12 in vivo can also be associated with significant systemic toxicity.

The IL-12 family of cytokines comprises heterodimeric cytokines that include IL-2, IL-23, IL-27, IL-35, and IL-39, as well as IL-12. The biologically active heterodimeric form of IL-12 comprises two subunits, p35 (alpha subunit) and p40 (beta subunit), linked covalently) by a disulphide bond (second linker). The IL-12 heterodimer is a 70 kDa protein, and can be referred to as IL-12p70. IL-12 is mainly produced by activated antigen-presenting cells (such as DCs, macrophages, monocytes, and B cells). IL-12 production is a tightly controlled process, and is regulated mainly at the transcriptional level. In addition to its cytotoxic activity, IL-12 is antiangiogenic, reducing the vasculature of cancer cells. The antimitogenic activity may be an effect of IL-12 activation recruiting kinases, which, upon dimerization and nuclear translocation, ultimately lead to IFN-gamma production. In clinical trials, systemic IL-12 can be toxic. Local IL-12 delivery and the use of immunostimulatory monoclonal antibodies can generate synergistic results.

### Protease Cleavage Site

In the present disclosure, the effector molecule E is connected to the transmembrane protein, or portion thereof, via a first peptide linker, “L”. The peptide cleavable linker is cleavable by a protease expressed in an isolated cell or in vivo in a cancer cell. Advantageously, the peptide protease cleavable linker L (first linker) is a sequence that is cleaved by a protease that is expressed in cancer cells or the TME. For example, the linker L may be cleavable by a matrix metalloprotease, such as MMP2.

In certain embodiments, the linker L comprises one or more protease-binding substrate sequence selected from ISSGLLSGRSDNH (SEQ ID NO. 8), VPLSLYSGGGISSGLLSGRSDNH (SEQ ID NO. 9), HPVGLLAR (SEQ ID NO. 10), SGRSA (SEQ ID NO. 11), and LAQAVRSS (SEQ ID NO. 12).

In the examples described herein, a non-cleavable Gly-Ser peptide linker is G4Sx3 (TM) (SEQ ID NO: 1). Linker L1 is a longer non-cleavable Gly-Ser type linker, G4Sx4 (SEQ ID NO: 2). Gly-Ser type linkers are commonly used as peptide linkers in fusion proteins (see Klein et al (2014). Design and characterization of structured protein linkers with different flexibilities. Protein Engineering, Design & Selection, vol. 27 no. 10 pp. 325-330). Gly-Ser linkers are also described in Linkers L2, L3, L4, L4s, and L5 are protease-cleavable linkers, comprising protease substrate sequences inserted between Gly-Ser peptide sequences.

L2, GGGGSISSGLLSGRSDNHGGGGS (SEQ ID NO: 3), is a peptide linker cleavable by the proteases urokinase plasminogen activator (uPA), matriptase (also known as MT-SP1), and matrix metalloproteinase (MMP). The bolded and underlined amino acids in the sequence (as well as in the other sequences) are the protease cleavable peptide portion of the linker. The sequence LSGRSDNH (SEQ ID NO: 13) is cleavable by uPA and/or matriptase (see for e.g. US 2017/0204139, WO 2022/178753, WO 2022/178751, WO 2019/173382). The sequence ISSGLLSS (SEQ ID NO: 14) is cleavable by MMP (see for e.g. WO 2019/173382, US 2017/0204139).

L3, GGGGSVPLSLYSGGGISSGLLSGRSDNHGGGGS (SEQ ID NO: 4) is a peptide linker comprising MMP9+MMP/uPA/Matriptase (matriptase is also known as MT-SP1) cleavable sequences. The sequence VPLSLYSG (SEQ ID NO: 16) is cleavable by MMP, especially MMP2 and MMP9 (see for e.g. WO 2021/016599, WO 2021/016640, WO 2020/069398). The sequence LSGRSDNH (SEQ ID NO: 13) is cleavable by uPA and/or matriptase (see for e.g. US 2017/0204139, WO 2022/178753, WO 2022/178751, WO 2019/173382). The sequence ISSGLLSS (SEQ ID NO: 14) is cleavable by MMP (see for e.g. WO 2019/173382, US 2017/0204139).

L4, GGGGSHPVGLLARGGGHPVGLLARGGSGRSAGGSGRSAGGGGS (SEQ ID NO: 5), is a surface MMP2+uPA, which is a cell surface TNF prodrug cleavable linker. The sequence HPVGLLAR (SEQ ID NO: 10) is a substrate for MMP2 (see for e.g. WO 2021/016640). L4 has two repeats of HPVGLLAR. The sequence SGRSA (SEQ ID NO: 11) is cleavable by uPA and/or matriptase (see for e.g. WO 2022/178753, WO 2022/178751, WO 2021/149697, WO 2021/016599, WO 2021/016640, WO 2020/069398, US 2020/0207846).

L4s has only one repeat of HPVGLLAR (SEQ ID NO: 10), and is cleavable by MMP2.

L5, GGGGSLAQAVRSSGGGGS (SEQ ID NO: 7), comprises an ADAM/TACE/CD156q (i.e. TNF-alpha converting enzyme) cleavage site. The sequence LAQAVRSS (SEQ ID NO: 12) is a substrate for ADAM/TACE/CD156q.

In preferred embodiments, the protease cleavage site linker L is selected from the peptide sequences in Table A, where the bolded sequences are protease-binding substrates.

TABLE A
Protease cleavable linkers

| SEQ | | |
|---|---|---|
| ID | Linker | |
| NO | “L” | Peptide sequence |
| SEQ | L2 | GGGGSISSGLLSGRSDNHGGGGS |
| ID | | |
| NO: 3 | | |
| SEQ | L3 | GGGGSVPLSLYSGGGISSGLLSGR |
| ID | | SDNHGGGGS |
| NO: 4 | | |
| SEQ | L4 | GGGGSHPVGLLARGGGHPVGLLAR |
| ID | | GGSGRSAGGSGRSAGGGGS |
| NO: 5 | | |
| SEQ | L4s | GGGGSHPVGLLARGGGGS |
| ID | | |
| NO: 6 | | |
| SEQ | L5 | GGGGSLAQAVRSSGGGGS |
| ID | | |
| NO: 7 | | |

### Proteases

Of particular interest are proteases that are preferentially expressed by tumor cells, or the expression of which is dysregulated in cancer cells and the TME.

In some embodiments, isolated cells expressing the chimeric protein of the disclosure contain endogenous or exogenous proteases that cleave the protease cleavable linker.

Metalloproteases, a family of Zn2+ binding protease homologues, are associated with numerous cancer-related functions. The most important metalloproteases that are active in the tumor microenvironment are the matrix metalloproteases (MMPs), a disintegrin and metalloproteases (ADAMs), and a disintegrin and metalloproteinase with thrombospondin motifs (ADAMTSs).

Elevated activity of MMPs has been detected in almost all types of cancer. MMPs target a broad range of extracellular matrix (ECM) proteins, thus contributing to cancer development, progression, invasive growth, and spread of cancer cells. The proteolytic action of MMPs on ECM scaffolding protein changes the composition, structure, and function of the ECM. MMPS may be soluble (e.g. MMP-1, -2, -7, -8, -9, 10, -11, -13, -26), while other MMPs are membrane-bound, anchored to the cell surface by a GPI-anchor (e.g. MMP-14, -15, -16, -17, -24, and -25).

ADAMs are transmembrane proteases, and are involved in cell proliferation, adhesion and migration. ADAMs play a significant role in the complexity of proteolysis in the tumor. ADAMs generally target the extracellular domains of transmembrane proteins, both type and type II, contributing to the cleavage of cell adhesion molecules, shedding of cell surface receptors, and, interestingly for the present disclosure, the maturation of cytokines and chemokines. ADAM-10 and ADAM-17 (also known as tumor necrosis-alpha converting enzyme, TACE) have primarily been shown to be involved in cancer. ADAM-10 processes and activates the epidermal growth factor (EGFR) ligands, cleaves E-cadherin (suggesting effects on signaling), and cleaves the CD44 adhesion molecule from the cell surface. ADAM-10 expression has been shown to correlate with invasive growth of several cancers. ADAM-17 contributes to the release of soluble TNF-alpha. As discussed above, TNF-alpha may have antiangiogenic effects. ADAM-17 also activates IL-6/ERK signaling, and it can release the EGF receptor, and shed adhesion molecules, such as CD44.

ADAMTSs are largely responsible for degrading structural ECM proteins. Different ADAMTSs may be involved in cancer development and progression, and can promote tumor development, but some ADAMTSs may also have tumor suppressor effects.

Other proteases that may play a role in cancer are serine proteases, cysteine proteases, aspartic proteases, and threonine proteases. For example, urokinase-type plasminogen activator (uPA) is a trypsin-like protease that catalyzes the activation of urokinase-type plasminogen activator receptor (uPAR), and cleaves several components of the ECM. Matriptase is a membrane-anchored serine protease that is involved in the serine protease-growth factor signaling axis, and has been studied as a potential diagnostic marker for cancer.

### N-Terminus Signal Peptide

In some embodiments, the chimeric protein disclosed herein further comprises a signal peptide at the N-terminus of the chimeric protein. The signal peptide directs newly synthesized proteins destined for secretion to membrane localization to the proper protein processing pathways. The signal peptide may comprise a native signal peptide—that is, a signal peptide that is native to the effector molecule. Alternatively, the signal peptide may comprise a non-native signal peptide. Suitable non-native signal peptides include IL-12, IL-2, optimized IL-2, trypsinogen-2, Gaussia luciferase, CD5, human IgKVII, murine IgKVII, VSV-G, prolactin, serum albumin preprotein, azurocidin preprotein, osteonectin, CD33, IL-6, IL-8, CCL2, TIMP2, VEGFB, osteoprotegrin, serpin E1, GROalpha, CXCL12, IL-21, CD8, NKG2D TNFR2 and GMCSF.

### Transmembrane Proteins

Transmembrane proteins are integral membrane proteins that span through the entire cell wall lipid bilayer. Transmembrane proteins have three regions: extracellular domain, transmembrane domain (TM), and intracellular domain (CD). Structurally, there are several types of transmembrane proteins: (1) a single transmembrane alpha-helix, or biopic membrane protein, that spans the membrane only once; (2) a polytopic transmembrane alpha-helix protein that spans the membrane more than once (e.g. 7-transmembrane proteins such as G-protein coupled receptors); and polytopic beta-sheet protein. Transmembrane proteins are also classified based on topology, that is, the position of the N- and C-termini on different sides of the lipid bilayer—e.g. Types I, II, III, and IV single-pass molecules. Type I transmembrane proteins are single-pass transmembrane proteins, having extracellular (luminal) N-termini, and cytoplasmic C-termini for a cell. Type II transmembrane proteins are single-pass transmembrane proteins, having extracellular (luminal) C-termini, and cytoplasmic N-termini for a cell.

The transmembrane protein, TM, of the disclosure is preferably a transmembrane domain portion of a transmembrane protein. In certain embodiments, the TM is derived from PDGFR-beta, CD8, CD28, CD3 zeta-chain, CD4, 4-1BB, OX40, ICOS, CTLA-4, PD-1, LAG-3, 2B4, LNGFR, NKG2D, EpoR, TNFR2, B7-1, and BTLA. In some embodiments the TM is further linked to a cytoplasmic domain, to give a TMCD construct. The cytoplasmic domain is derived from PDGFR-beta, CD8, CD28, CD3 zeta-chain, CD4, 4-1BB, OX40, ICOS, CTLA-4, PD-1, LAG-3, 2B4, LNGFR, NKG2D, EpoR, TNFR2, B7-1, and BTLA. In certain embodiments, either the TM or the CD, or both the TM and CD, are derived from B7-1 transmembrane protein.

PDGFR-beta (platelet-derived growth factor receptor-beta) is a type I transmembrane protein. PDGFR-beta is expressed is several tumors, and its expression is correlated with tumor growth, invasiveness, drug resistance, and poor clinical outcomes.

CD8 (cluster of differentiation 8) is a dimeric type I transmembrane protein expressed on cytotoxic T-cells, as well as natural killer (NK) cells. CD8+ cytotoxic T cells have been shown to be useful in anticancer therapy.

CD28 is a type I transmembrane protein expressed on most naïve CD4 and CD8 T cells. CD28 is a costimulatory molecule on T cells. CD28 has been studied in cancer immunotherapy.

CD3 zeta chain (also known as CD247) is part of the T-cell receptor (TCR) CD3 complex. CD3 zeta chain is a transmembrane protein in the TCR complex.

CD4 is a monomeric transmembrane protein found on immune cells, including T helper cells, monocytes, macrophages, and dendritic cells. It is a co-receptor for TCR.

4-1BB is a glycosylated type I membrane protein. 4-1BB is not expressed on naïve T cells, but is upregulated after T cell binding with the MHC: peptide on antigen presenting cells. 41-BBL is the binding partner for 4-1BB, and is a type II membrane protein of the tumor necrosis factor superfamily.

OX40 is a type I transmembrane glycoprotein. It is expressed on several types of cells, include CD4+ and CD8+ T cells.

ICOS (also known as CD278) is a type I transmembrane glycoprotein. ICOS belongs to the CD28 family of co-stimulatory immunoreceptors, and is expressed on antigen presenting cells.

CTLA-4 (cytotoxic T-lymphocyte-associated protein 4) is a type I transmembrane protein expressed on activated T cells. Blockade of CTLA-4 can be a treatment for cancer.

PD-1 (programmed cell death protein 1; also known as CD279) is a type I transmembrane protein expressed on T and B cells. Blockade of PD-1 can be a treatment for cancer.

LAG-3 (lymphocyte activation gene 3; also known as CD223) is a type I transmembrane protein. It is expressed as a dimer or as an oligomer on activated CD4+ and CD8+ Tcells, NK cells, B cells, regulatory T cells, Tr1 cells, natural killer cells, and plasmacytoid dendritic cells. It is upregulated on exhausted T cells in cancer.

2B4 is a type I transmembrane protein expressed on natural killer (NK) cells, and can play a role in tumor formation.

LNGFR (low-affinity nerve growth factor receptor; also known as p75 neurotrophin receptor (p75NTR)) is a type I transmembrane protein. LNGFR can be a marker for cancer stem cells. Use of LNGFR as a spacer in CAR T cells is also of interest.

NKG2D (natural killer cell 2D receptor) is expressed on NK cells and T cells. NKG2D is a type II transmembrane glycoprotein. NKG2D binds to a ligand expressed in tumor cells.

EpoR (erythropoietin receptor) is a member of the cytokine type I transmembrane receptor family. Splice variants of EpoR have been detected in tumors.

TNFR2 (tumor necrosis factor receptor type II) is expressed in several types of tumor cells, as well as immune cells such as regulatory T cells and myeloid-derived suppressing cells, and is enriched in the TME. TNFR2 is a type I transmembrane protein.

B7-1 is type I transmembrane protein. B7-1 is a ligand of the CD28 family of receptors, and is expressed on activated antigen-presenting cells, T lymphocytes, and tumor cells. B7-1 is involved in mediating the dynamic interactions between cancer cells and the host immune system.

BTLA (B- and T-lymphocyte attenuator) is an immune regulatory receptor expressed on B cells, T cells, and all mature lymphocyte cells. BTLA is a type I transmembrane protein.

### Isolated Cells

In certain embodiments, the chimeric proteins of the disclosure are expressed in isolated cells. Suitable isolated cells include a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) 15 cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell. In some aspects, the isolated cell is a Natural Killer (NK) 20 cell.

Isolated cells also include tumor cells. Suitable tumor cells include a bladder tumor cell, a brain tumor cell, a breast tumor cell, a cervical tumor cell, a colorectal tumor cell, an esophageal tumor cell, a glioma cell, a kidney tumor cell, a 25 liver tumor cell, a lung tumor cell, a melanoma cell, an ovarian tumor cell, a pancreatic tumor cell, a prostate tumor cell, a skin tumor cell, a thyroid tumor cell, and a uterine tumor cell.

In some embodiments, when the chimeric protein of the present disclosure is expressed in a cell, the effector molecule E is tethered to a cell membrane of the cell. The transmembrane domain TM is integral to the cell membrane, and the effector molecule E is bonded to the TM via the protease cleavage site L.

In some embodiments, when the chimeric protein of the disclosure is expressed in a cell that expresses a protease capable of cleaving the protease cleavage site L the effector molecule E is released from the cell membrane.

In some embodiments, the protease expressed on the cell membrane is endogenous to the cell. In other embodiments, the cell has been engineered to express a protease that is exogenous to the cell. Preferably, the protease is one that is expressed by cancer cells.

Advantageously, the cell comprises the chimeric protein of the disclosure, the nucleic acid molecule of the disclosure (described below), or the expression vector of the disclosure (described below).

As used herein, a “stable expression” of a transfected or transduced gene in a host cell means the integration of said gene in the genome of said host cell and as a result, is able to express the transfected genetic material.

### Nucleic Acids

The present disclosure also includes recombinant nucleic acids that encode the chimeric proteins of the disclosure. By “recombinant,” as used herein, is meant the modification of a nucleic acid or amino acid sequence, resulting in a product that is not found in nature. When made in reference to a nucleic acid construct, the term refers to a molecule that is comprised of nucleic acid sequences that are joined together or produced by means of molecular biological techniques. The term “recombinant” when made in reference to a protein or a polypeptide refers to a protein or polypeptide molecule that is expressed using a recombinant nucleic acid construct created by means of molecular biological techniques. Recombinant nucleic acid constructs may include a nucleotide sequence which is ligated to, or is manipulated to become ligated to, a nucleic acid sequence to which it is not ligated in nature, or to which it is ligated at a different location in nature. Referring to a nucleic acid construct as “recombinant” therefore indicates that the nucleic acid molecule has been manipulated using genetic engineering, i.e. by human intervention. Recombinant nucleic acid constructs may for example be introduced into a host cell by any suitable means described herein or known in the art. Such recombinant nucleic acid constructs may include sequences derived from the same host cell species or from different host cell species, which have been isolated and reintroduced into cells of the host species. Recombinant nucleic acid construct sequences may become integrated (“stably incorporated”) into a host cell genome, for example the genome of an oncolytic virus, either as a result of the original transformation of the host cells, or as the result of subsequent recombination and/or repair events.

The nucleic acid may be an RNA sequence. As used herein, “RNA” refers to a molecule comprising one or more ribonucleotide residues. A “ribonucleotide” is a nucleotide with a hydroxyl group at the 2′ position of the beta-D-ribofuranose moiety. The term “RNA” includes double-stranded RNA, single-stranded RNA, isolated RNA (e.g. partially purified RNA), essentially pure RNA, synthetic RNA, and recombinantly produced RNA. The term “RNA” also refers to modified RNA that differs from naturally-occurring RNA by the addition, deletion, substitution and/or alteration of one or more nucleotides, such as non-naturally occurring nucleotides or chemically synthesized nucleotides or deoxynucleotides. In other embodiments, the nucleic acid may be a DNA sequence.

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

The present disclosure also provides a nucleic acid molecule comprising an expression vector (expression construct, expression cassette) wherein the expression cassette comprises a promoter and an exogenous polynucleotide sequence encoding the chimeric protein. In some embodiments, the expression cassette may comprise a polynucleotide that encodes a reporter gene, such as mCherry or an enhanced green fluorescent protein (EGFP).

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes. A “promoter/regulatory sequence” means a nucleic acid sequence which is required for expression of a gene product operably linked to the promoter/regulatory sequence. This sequence may be the core promoter sequence, or it may also include an enhancer sequence and other regulatory elements which are required for expression of the gene product.

As used herein, “constitutive promoter” is one that is active in all tissues or particular tissues at all developing stages. Examples of constitutive promoters include, but are not limited to, cytomegalovirus immediate early promoter (CMV), simian virus (SV40) promoter, adenovirus major late (MLP) promoter, Rous sarcoma virus (RSV) promoter, elongation factor-alpha (EF1a) promoter, ubiquitin promoters, actin promoters, tubulin promoters, immunoglobulin promoters, functional fragments thereof, or combinations thereof. Other suitable promoters are known to one of skill in the art.

As used herein, “inducible promoter” is a nucleotide sequence which, when operably linked with a polynucleotide which encodes or specifies a gene product, causes the gene product to be produced in a cell substantially only when an inducer which corresponds to the promoter is present in the cell. Examples of inducible promoters include, for example, chemically regulated promoters and physically-regulated promoters. Examples of inducible promoters include, but are not limited to, those inducible by heat shock, light, chemicals, peptides, metals, steroids, antibiotics, or alcohol. Chemically regulated promoters include, for example, alcohol-regulated promoters (e.g., an alcohol dehydrogenase (alcA) gene promoter), tetracycline-regulated promoters (e.g., a tetracycline-responsive promoter, a tetracycline operator sequence (tetO), a tet-On promoter, or a tet-Off promoter), steroid regulated promoters (e.g., a rat glucocorticoid receptor, a promoter of an estrogen receptor, or a promoter of an ecdysone receptor), or metal-regulated promoters (e.g., a metalloprotein promoter). Physically regulated promoters include, for example temperature-regulated promoters (e.g., a heat shock promoter) and light-regulated promoters (e.g., a light-inducible promoter or a light-repressible promoter).

In some embodiments the promoter may be tissue specific. Tissue-specific promoters can be, for example, neuron-specific promoters, glia-specific promoters, muscle cell-specific promoters, heart cell-specific promoters, kidney cell-specific promoters, bone cell-specific promoters, endothelial cell-specific promoters, or immune cell-specific promoters (e.g., a B cell promoter or a T cell promoter).

As used herein, a “tissue specific promoter” is a nucleotide sequence which, when operably linked with a polynucleotide which encodes or specifies a gene product, causes the gene product to be produced in a cell substantially only if the cell is a cell of the tissue type corresponding to the promoter. Tissue specific promoters include, but are not limited to, CAG, SYN1, CMV, NSE, CBA, PDGF, SV40, RSV, LTR, SV40, dihydrofolate reductase promoter, beta-actin promoter, PGK, EF1alpha, GRK, MT, MMTV, TY, RU486, RHO, RHOK, CBA, chimeric CMV-CBA, MLP, RSV, ubiquitin promoters, actin promoters, tubulin promoters, immunoglobulin promoters, functional fragments thereof, etc.

Tissue-specific promoters can be, for example, neuron-specific promoters, glia-specific promoters, muscle cell-specific promoters, heart cell-specific promoters, kidney cell-specific promoters, bone cell-specific promoters, endothelial cell-specific promoters, or immune cell-specific promoters (e.g., a B cell promoter or a T cell promoter).

In a preferred embodiment of the present disclosure, the promoter is selected from the group consisting of a constitutive promoter, an inducible promoter, a tissue-specific promoter, and a synthetic promoter.

The term “wild type” includes entities having a structure and/or activity as found in a normal (as contrasted with mutant, diseased, altered, or so forth) state or context. Wild type genes and polypeptides often exist in multiple different forms (e.g., alleles).

The term “endogenous sequence” refers to a nucleic acid sequence that occurs naturally within a cell or eukaryotic organism (e.g., animal, non-human animal, mammal, or non-human mammal).

“Exogenous” molecules or sequences include molecules or sequences that are not normally present in a cell in that form. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two segments that do not naturally occur together in the same molecule. For example, the term “heterologous,” when used with reference to segments of a nucleic acid or segments of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by sequences not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

The term “locus” refers to a specific location of a gene (or significant sequence), DNA sequence, polypeptide-encoding sequence, or position on a chromosome of the genome of an organism. For example, a “Ttr locus” may refer to the specific location of a Ttr gene, Ttr DNA sequence, TTR-encoding sequence, or Ttr position on a chromosome of the genome of an organism that has been identified as to where such a sequence resides. A “Ttr locus” may comprise a regulatory element of a Ttr gene, including, for example, an enhancer, a promoter, 5′ and/or 3′ untranslated region (UTR), or a combination thereof.

The term “gene” refers to a DNA sequence in a chromosome that codes for a product (e.g., an RNA product and/or a polypeptide product) and includes the coding region interrupted with non-coding introns and sequence located adjacent to the coding region on both the 5′ and 3′ ends such that the gene corresponds to the full-length mRNA (including the 5′ and 3′ untranslated sequences). The term “gene” also includes other non-coding sequences including regulatory sequences (e.g., promoters, enhancers, and transcription factor binding sites), polyadenylation signals, internal ribosome entry sites, silencers, insulating sequence, and matrix attachment regions. These sequences may be close to the coding region of the gene (e.g., within 10 kb) or at distant sites, and they influence the level or rate of transcription and translation of the gene.

The term “allele” refers to a variant form of a gene. Some genes have a variety of different forms, which are located at the same position, or genetic locus, on a chromosome. A diploid organism has two alleles at each genetic locus. Each pair of alleles represents the genotype of a specific genetic locus. Genotypes are described as homozygous if there are two identical alleles at a particular locus and as heterozygous if the two alleles differ.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

The chimeric proteins disclosed herein can comprise one or more transcriptional activation domains. Transcriptional activation domains include regions of a naturally occurring transcription factor which, in conjunction with a DNA-binding domain, can activate transcription from a promoter by contacting transcriptional machinery either directly or through other proteins such as coactivators. Transcriptional activation domains also include functional fragments or variants of such regions of a transcription factor and engineered transcriptional activation domains that are derived from a native, naturally occurring transcriptional activation domain or that are artificially created or synthesized to activate transcription of a target gene. A functional fragment is a fragment that is capable of activating transcription of a target gene when operably linked to a suitable DNA-binding domain. A functional variant is a variant that is capable of activating transcription of a target gene when operably linked to a suitable DNA-binding domain.

A specific transcriptional activation domain for use in the chimeric proteins disclosed herein comprises a VP64 transcriptional activation domain or a functional fragment or variant thereof. VP64 is a tetrameric repeat of the minimal activation domain from the herpes simplex VP16 activation domain. Other examples of transcriptional activation domains include herpes simplex virus VP16 transactivation domain, VP64 (quadruple tandem repeat of the herpes simplex virus VP16), a NF-κB p65 (NF-κB trans-activating subunit p65) activation domain, a MyoDI transactivation domain, an HSF1 transactivation domain (transactivation domain from human heat-shock factor 1), RTA (Epstein Barr virus R transactivator activation domain), a SETT/9 transactivation domain, a p53 activation domain 1, a p53 activation domain 2, a CREB (cAMP response element binding protein) activation domain, an E2A activation domain, an NFAT (nuclear factor of activated T-cells) activation domain, and functional fragments and variants thereof. See, e.g., US 2016/0298125, US 2016/0281072, and WO 2016/049258, each of which is herein incorporated by reference in its entirety for all purposes. Other examples of transcriptional activation domains include Gcn4, MLL, Rtg3, Gln3, Oaf1, Pip2, Pdr1, Pdr3, Pho4, Leu3, and functional fragments and variants thereof. See, e.g., US 2016/0298125, herein incorporated by reference in its entirety for all purposes. Yet other examples of transcriptional activation domains include Sp1, Vax, GATA4, and functional fragments and variants thereof. See, e.g., WO 2016/149484, herein incorporated by reference in its entirety for all purposes. Other examples include activation domains from Oct1, Oct-2A, AP-2, CTF1, P300, CBP, PCAF, SRC1, PvALF, ERF-2, OsGAI, HALF-1, C1, AP1, ARF-5, ARF-6, ARF-7, ARF-8, CPRF1, CPRF4, MYC-RP/GP, and TRAB 1PC4, and functional fragments and variants thereof. Sec, e.g., US 2016/0237456, EP3045537, and WO 2011/146121, each of which is incorporated by reference in its entirety for all purposes. Additional suitable transcriptional activation domains are also known. See, e.g., WO 2011/146121, herein incorporated by reference in its entirety for all purposes.

### Vector

In some embodiments, the chimeric proteins of the disclosure, or nucleic acids expressing the chimeric proteins of the disclosure, are expressed in the isolated cells via transduction with a vector.

As used herein, “vector” is a composition of matter which comprises an isolated nucleic acid encoding the chimeric protein of the disclosure, and which can be used to deliver the isolated nucleic acid to the interior of a cell. Vectors include, but are not limited to, linear polynucleotides, polynucleotides associated with ionic or amphiphilic compounds, plasmids, and viruses. The term “vector” includes an autonomously replicating plasmid or a virus. “Vector” may also include non-plasmid and non-viral compounds which facilitate transfer of nucleic acid into cells, such as, for example, polylysine compounds liposomes, lipid nanoparticles, non-lipid nanoparticles, and the like. In preferred embodiments, the vector is a viral vector. Viruses that can be engineered to be used as viral vectors include, but are not limited to, retrovirus, lentivirus, adenovirus, adeno-associated virus (AAV), alphavirus, flavivirus, rhabdovirus, paramyxovirus, picornavirus, poxvirus, and herpesvirus. Of these, oncolytic viral vectors are preferred, and include, but are not limited to, adenovirus, herpesvirus, alphavirus, flavivirus, rhabdovirus, paramyxovirus, and cytomegalovirus. In some embodiments, the recombinant oncolytic virus may be an oncolytic RNA virus, such as a vesicular stomatitis virus (VSV), Maraba Virus, Newcastle Disease Virus, Poliovirus, Measles Virus or Reovirus. In some embodiments, the recombinant oncolytic virus may be an oncolytic DNA virus, such as a Vaccinia Virus (VV), Herpes Simplex Virus (HSV), or Adenovirus. In some embodiments, the recombinant oncolytic virus may be VSV-CXCL12, VV-CXCL12, VSV-CXCL13, VSV-M51R-VacE3L, VV-CXCL13, VSV-CXCL10 or VV-CXCL10. Preferably, the viral vector is an oncolytic virus.

Adeno-associated virus (AAV) is a small, replication-deficient parvovirus. AAV is about 20-24 nm long, with a density of about 1.40-1.41 g/cc. AAV contains a single-stranded linear genomic DNA molecule approximately 4.7 kb in length. The single-stranded AAV genomic DNA can be either a plus strand, or a minus strand. AAV contains two open reading frames, Rep and Cap, flanked by two 145 base inverted terminal repeats (ITRs). AAVs contain a single intron. Cis-acting sequences directing viral DNA replication (Rep), encapsidation/packaging and host cell chromosome integration are contained within the ITRs. Three AAV promoters, p5, p19, and p40 (named for their relative map locations) drive the expression of the two AAV internal open reading frames encoding rep and cap genes. The p5 and p19 are the rep promoters. When coupled with the differential splicing of the single AAV intron, the two rep promoters result in the production of four rep proteins (rep 78, rep 68, rep 52, and rep 40) from the rep gene. The rep proteins have multiple enzymatic properties that are responsible for replicating the viral genome. The cap gene is expressed from the p40 promoter, and encodes the three capsid proteins VP1, VP2, and VP3. Alternative splicing and non-consensus translational start sites are responsible for the production of the three related capsid proteins. A single polyadenylation site is located at map position 95 of the AAV genome. Muzyczka reviews the life cycle and genetics of AAV (Muzyczka, Current Topics in Microbiology and Immunology, 158:97-129 (1992)).

AAV infection is non-cytopathic in cultured cells. Natural infection of humans and other animals is silent and asymptomatic (does not cause disease). Because AAV infects many mammalian cells, there is the possibility of targeting many different tissues in vivo. In addition to dividing cells, AAV transduces slowly dividing and non-dividing cells, and can persist essentially for the lifetime of those cells as a transcriptionally active nuclear episome (i.e. extrachromosomal element). The AAV proviral genome is infectious as cloned DNA in plasmids, which makes construction of recombinant genomes possible. Moreover, because the signals directing AAV replication, genome encapsidation, and integration are all contained with the ITRs of the AAV genome, some or all of the approximately 4.3 kb of the genome, encoding replication and structural capsid proteins (rep-cap) are contained within the ITRs of the AAV genome, can be replaced with heterologous DNA, such as a gene cassette containing a promoter, a DNA of interest, and a polyadenylation signal. The rep and cap proteins may be provided in trans.

Several AAV serotypes have been identified, differing in their tropism (type of cell that they infect). Serotype AAV1 shows tropism to the following tissues: CNS; heart; retinal pigment epithelium (RPE); and skeletal muscle. Serotype AAV2 shows tropism to the following tissues: CNS; kidney; photoreceptor cells; and RPE. Serotype AAV4 shows tropism to the following tissues: CNS; lung; and RPE. Serotype AAV5 shows tropism to the following tissues: CNS; lung; photoreceptor cells; and RPE. Serotype AAV6 shows tropism to the following tissues: lung; and skeletal muscle. Serotype AAV7 shows tropism to the following tissues: liver; and skeletal muscle. Serotype AAV8 shows tropism to the following tissues: CNS; heart; liver; pancreas; photoreceptor cells; RPE; and skeletal muscle. Serotype AAV9 shows tropism for the following tissues: CNS; heart; liver; lung; and skeletal muscle. The tropism of AAV viruses may be related to the variability of the amino acid sequences of the capsid protein, which may bind to different functional receptors present on different types of cells.

For example, it has recently been shown that including a human rhodopsin kinase (hGRK1) promoter in an AAV5 vector results in rod- and cone-specific expression in the primate retina (Boye, et al., Human Gene Therapy, 23:1101-1115 (October 2012) (DOI: 10.1089/hum.2012.125)).

It has also recently been shown that AAV virions with altered capsid proteins may impart greater tissue specific infectivity. For example, AAV6 with a variant capsid protein shows increased infectivity of retinal cells, compared to wild-type AAV capsid protein (U.S. Pat. No. 8,663,624). A variant capsid protein comprising a peptide insertion between two adjacent amino acids corresponding to amino acids 570 ad 611 of VP1 of AAV2, or the corresponding position in a capsid protein of another AAV serotype, confers increased infectivity of retinal cells, compared to wild-type AAV (U.S. Pat. No. 9,193,956).

Lentivirus is a genus of retroviruses that cause chronic and deadly diseases characterized by long incubation periods, in the human and other mammalian species. The best known lentivirus is the human immunodeficiency virus (HIV), which causes AIDS. Lentiviruses are also hosted in apes, cows, goats, horses, cats, and sheep. Recently, lentiviruses have been found in monkeys, lemurs, Malayan flying lemur (neither a true lemur nor a primate), rabbits, and ferrets. Lentiviruses and their hosts have worldwide distribution. Lentiviruses can integrate a significant amount of viral cDNA into the DNA of the host cell and can efficiently infect non-dividing cells, so they are one of the most efficient methods of gene delivery. Lentiviruses can become endogenous (ERV), integrating their genome into the host germline genome, so that the virus is henceforth inherited by the host's descendants.

Lentivirus is primarily a research tool used to introduce a gene product into in vitro systems or animal models. Conversely, lentivirus is also used to stably over-express certain genes, thus allowing researchers to examine the effect of increased gene expression in a model system.

Another common application is to use a lentivirus to introduce a new gene into human or animal cells. For example, a model of mouse hemophilia is corrected by expressing wild-type platelet-factor VIII, the gene that is mutated in human hemophilia. Lentiviral infection has advantages over other gene-therapy methods including high-efficiency infection of dividing and non-dividing cells, long-term stable expression of a transgene, and low immunogenicity. Lentiviruses have also been successfully used for transduction of diabetic mice with the gene encoding PDGF (platelet-derived growth factor), a therapy being considered for use in humans. Finally, lentiviruses have been also used to elicit an immune response against tumor antigens. These treatments, like most current gene therapy experiments, show promise but are yet to be established as safe and effective in controlled human studies. Gammaretroviral and lentiviral vectors have so far been used in more than 300 clinical trials, addressing treatment options for various diseases.

Vesicular stomatitis virus Indiana strain is a prototypical strain of the genus vesiculovirus, which belongs to the rhabdovirus family—the term VSV refers to this strain. Other major serotypes include Cocal, Vesicular stomatitis virus New Jersey strain, Chandipura, Maraba, and Piry viruses. VSV is enveloped, and has a negative-sense RNA genome. The single-stranded, negative-sense RNA genome of VSV encodes five structural proteins: nucleoprotein (N), phosphoprotein (P), matrix protein (M; responsible for formation of the viral core and anchoring of the glycoprotein to the viral membrane enabling the formation of glycoprotein homotrimers), glycoprotein (G; controls receptor recognition, cell entry, and viral fusion), and the viral polymerase. Because the glycoprotein controls receptor recognition, cell entry, and viral fusion it is the primary target for the primary humoral response. The glycoprotein is referred to as VSV.G.

Most virus vectors based on enveloped viruses are pseudotypes, wherein their envelopes are not encoded by their genome, but rather are derived from related viruses. VSV.G is one of the most widely used viral envelopes because of its broad tropism (its primary receptor LDLR is widely expressed in most tissues), thermal, and physical stability. For example, lentiviral vectors pseudotyped with VSV.G are used in gene therapy applications.

VSV has been used as a vector for vaccine therapy. However, currently, there are two problems with this approach. VSV.G generates a potent neutralizing antibody response, and therefore vaccine vectors expressing VSV.G cannot be re-administered. Also, VSV is a neurotropic virus, and may have off-target neurotoxicity-related adverse effects.

VSV can be an oncolytic virus. The lack of anti-viral machinery in cancer cells enables oncolytic viruses to preferentially infect and replicate in cancer cells, and spread to other tumor tissues, killing them. VSV's onco-selectivity is a result of its sensitivity to type I interferon dependent cellular immune responses. Although VSV infection in healthy cells is reduced by a robust IFN-1 response, in cancer cells, parts of this immune pathway are missing or damaged. Thus, VSV preferentially replicates in cancerous tissue.

The viral vector may comprise an expression vector, expression construct, or expression cassette. The term “expression vector” or “expression construct” or “expression cassette” refers to a recombinant nucleic acid containing a desired coding sequence operably linked to appropriate nucleic acid sequences necessary for the expression of the operably linked coding sequence in a particular host cell or organism. Nucleic acid sequences necessary for expression in prokaryotes usually include a promoter, an operator (optional), and a ribosome binding site, as well as other sequences. Eukaryotic cells are generally known to utilize promoters, enhancers, and termination and polyadenylation signals, although some elements may be deleted and other elements added without sacrificing the necessary expression.

The expression cassettes disclosed herein can comprise other components as well. Such expression cassettes can further comprise a 3′ splicing sequence at the 5′ end of the expression cassette and/or a second polyadenylation signal following the coding sequence. The term 3′ splicing sequence refers to a nucleic acid sequence at a 3′ intron/exon boundary that can be recognized and bound by splicing machinery. An expression cassette can further comprise a selection cassette comprising, for example, the coding sequence for a drug resistance protein.

Examples of suitable selection markers include neomycin phosphotransferase (neo.sup.r), hygromycin B phosphotransferase (hyg.sup.r), puromycin-N-acetyltransferase (puro.sup.r), blasticidin S deaminase (bsr.sup.r), xanthine/guanine phosphoribosyl transferase (gpt), and herpes simplex virus thymidine kinase (HSV-k). Optionally, the selection cassette can be flanked by recombinase recognition sites for a site-specific recombinase. If the expression cassette also comprises recombinase recognition sites flanking a polyadenylation signal upstream of the coding sequence as described above, the selection cassette can be flanked by the same recombinase recognition sites or can be flanked by a different set of recombinase recognition sites recognized by a different recombinase.

An expression cassette can also comprise a nucleic acid encoding one or more reporter proteins, such as a fluorescent protein (e.g., a green fluorescent protein). Any suitable reporter protein can be used. For example, a fluorescent reporter protein can be used, or a non-fluorescent reporter protein can be used. Examples of fluorescent reporter proteins are provided elsewhere herein. Non-fluorescent reporter proteins include, for example, reporter proteins that can be used in histochemical or bioluminescent assays, such as beta-galactosidase, luciferase (e.g., Renilla luciferase, firefly luciferase, and NanoLuc luciferase), and beta-glucuronidase. An expression cassette can include a reporter protein that can be detected in a flow cytometry assay (e.g., a fluorescent reporter protein such as a green fluorescent protein) and/or a reporter protein that can be detected in a histochemical assay (e.g., beta-galactosidase protein). One example of such a histochemical assay is visualization of in situ beta-galactosidase expression histochemically through hydrolysis of X-Gal (5-bromo-4-chloro-3-indoyl-b-D-galactopyranoside), which yields a blue precipitate, or using fluorogenic substrates such as beta-methyl umbelliferyl galactoside (MUG) and fluorescein digalactoside (FDG).

The expression cassettes described herein can be in any form. For example, an expression cassette can be in a vector or plasmid. The expression cassette can be operably linked to a promoter in an expression construct capable of directing expression of a protein or RNA (e.g., upon removal of an upstream polyadenylation signal). Alternatively, an expression cassette can be in a targeting vector. For example, the targeting vector can comprise homology arms flanking the expression cassette, wherein the homology arms are suitable for directing recombination with a desired target genomic locus to facilitate genomic integration and/or replacement of endogenous sequence

### Pharmaceutical Compositions

The present disclosure provides a pharmaceutical composition comprising the chimeric protein of the disclosure, the nucleic acid encoding the chimeric protein of the disclosure, the expression vector that comprises the chimeric protein of the disclosure, or the isolated cell of the disclosure, and a pharmaceutically acceptable excipient. Pharmaceutically acceptable excipients include, but are not limited to, a water-insoluble diluent, a water-soluble diluent, a disintegrant, a binder, a wetting agent, a solubilizer, a glidant, a lubricant, and a granulating solvent.

### Lipid Nanoparticles

In preferred embodiments the pharmaceutical composition of the present disclosure is a lipid nanoparticle (LNP) pharmaceutical composition. LNP formulations are described in WO 2017/173054. Lipid nanoparticles (“LNPs”) are examples of vectors according to the present disclosure. LNPs are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as DSPC. In another example, the other components can comprise a helper lipid such as cholesterol, an optional neutral lipid such as DSPC, and a stealth lipid such as S010, S024, S027, S031, or S033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22 (9): 2227-2235 and WO 2017/173054 A1, each of which is herein incorporated by reference in its entirety for all purposes. In a preferred embodiment, the LNP includes a nucleic acid molecule coding for the chimeric protein of the present disclosure.

The lipid for encapsulation and endosomal escape can be a cationic lipid. The lipid can also be a biodegradable lipid, such as a biodegradable ionizable lipid. One example of a suitable lipid is Lipid A or LP01, which is (9Z,12Z)-3-((4,4-bis(octyloxy) butanoyl)oxy)-2-((((3-(diethylamino) propoxy-) carbonyl)oxy)methyl) propyl octadeca-9,12-dienoate, also called 3-((4,4-bis(octyloxy) butanoyl)oxy)-2-((((3-(diethylamino) propoxy) carbonyl-)oxy)methyl) propyl (9Z,12Z)-octadeca-9,12-dienoate. See, e.g., Finn et al. (2018) Cell Rep. 22 (9): 2227-2235 and WO 2017/173054 A1, each of which is herein incorporated by reference in its entirety for all purposes. Another example of a suitable lipid is Lipid B, which is ((5-((dimethylamino)methyl)-1,3-phenylene)bis(oxy))bis(octane-8,1-diyl)bis(decanoate), also called ((5-((dimethylamino)methyl)-1,3-phenylene)bis(oxy))bis(octane-8,1-diyl) bi-s (decanoate). Another example of a suitable lipid is Lipid C, which is 2-((4-(((3-(dimethylamino) propoxy) carbonyl)oxy) hexadecanoyl)oxy) propane-1-,3-diyl (9Z,9′Z,12Z,127)-bis(octadeca-9,12-dienoate). Another example of a suitable lipid is Lipid D, which is 3-(((3-(dimethylamino) propoxy) carbonyl)oxy)-13-(octanoyloxy)tridecyl 3-octylundecanoate. Other suitable lipids include heptatriaconta-6,9,28,31-tetraen-19-yl 4-(dimethylamino) butanoate (also known as [(6Z,9Z,28Z,31Z)-heptatriaconta-6,9,28,31-tetraen-19-yl] 4-(dimethylamino) butanoate or Dlin-MC3-DMA (MC3))).

Some such lipids suitable for use in the LNPs described herein are biodegradable in vivo. For example, LNPs comprising such a lipid include those where at least 75% of the lipid is cleared from the plasma within 8, 10, 12, 24, or 48 hours, or 3, 4, 5, 6, 7, or 10 days. As another example, at least 50% of the LNP is cleared from the plasma within 8, 10, 12, 24, or 48 hours, or 3, 4, 5, 6, 7, or 10 days.

Such lipids may be ionizable depending upon the pH of the medium they are in. For example, in a slightly acidic medium, the lipids may be protonated and thus bear a positive charge. Conversely, in a slightly basic medium, such as, for example, blood where pH is approximately 7.35, the lipids may not be protonated and thus bear no charge. In some embodiments, the lipids may be protonated at a pH of at least about 9, 9.5, or 10. The ability of such a lipid to bear a charge is related to its intrinsic pKa. For example, the lipid may, independently, have a pKa in the range of from about 5.8 to about 6.2.

Neutral lipids function to stabilize and improve processing of the LNPs. Examples of suitable neutral lipids include a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine or 1,2-distearoyl-sn-glycero-3-phosphocholine (DSPC), phosphocholine (DOPC), dimyristoylphosphatidylcholine (DMPC), phosphatidylcholine (PLPC), 1,2-diarachidonoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, diolcoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, 1-stearoyl-2-oleoyl-sn-glycero-3-phosphocholine (SOPC), and combinations thereof. For example, the neutral phospholipid may be selected from the group consisting of distearoylphosphatidylcholine (DSPC) and dimyristoyl phosphatidyl ethanolamine (DMPE).

Helper lipids include lipids that enhance transfection. The mechanism by which the helper lipid enhances transfection can include enhancing particle stability. In certain cases, the helper lipid can enhance membrane fusogenicity. Helper lipids include steroids, sterols, and alkyl resorcinols. Examples of suitable helper lipids suitable include cholesterol, 5-heptadecylresorcinol, and cholesterol hemisuccinate. In one example, the helper lipid may be cholesterol or cholesterol hemisuccinate.

Stealth lipids include lipids that alter the length of time the nanoparticles can exist in vivo. Stealth lipids may assist in the formulation process by, for example, reducing particle aggregation and controlling particle size. Stealth lipids may modulate pharmacokinetic properties of the LNP. Suitable stealth lipids include lipids having a hydrophilic head group linked to a lipid moiety.

The hydrophilic head group of stealth lipid can comprise, for example, a polymer moiety selected from polymers based on PEG (sometimes referred to as poly (ethylene oxide)), poly (oxazoline), poly(vinyl alcohol), poly (glycerol), poly (N-vinylpyrrolidone), polyaminoacids, and poly N-(2-hydroxypropyl) methacrylamide. The term PEG means any polyethylene glycol or other polyalkylene ether polymer. In certain LNP formulations, the PEG, is a PEG-2K, also termed PEG 2000, which has an average molecular weight of about 2,000 daltons. See, e.g., WO 2017/173054 A1, herein incorporated by reference in its entirety for all purposes.

The lipid moiety of the stealth lipid may be derived, for example, from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

As one example, the stealth lipid may be selected from PEG-dilauroylglycerol, PEG-dimyristoylglycerol (PEG-DMG), PEG-dipalmitoylglycerol, PEG-distearoylglycerol (PEG-DSPE), PEG-dilaurylglycamide, PEG-dimyristylglycamide, PEG-dipalmitoylglycamide, and PEG-distearoylglycamide, PEG-cholesterol (1-[8′-(Cholest-5-en-3 [beta]-oxy) carboxamido-3′,6′-dioxaoctanyl]carbamoyl- -[omega]-methyl-poly(ethylene glycol), PEG-DMB (3,4-ditetradecoxylbenzyl-[omega]-methyl-poly(ethylene glycol) ether), 1,2-dimyristoyl-sn-glycero-3-phosphoethanolamine-N-[methoxy (polyethylene glycol)-2000] (PEG2k-DMG), 1,2-distearoyl-sn-glycero-3-phosphoethanolamine-N-[methoxy (polyethylene glycol)-2000] (PEG2k-DSPE), 1,2-distearoyl-sn-glycerol, methoxypoly ethylene glycol (PEG2k-DSG), poly (ethylene glycol)-2000-dimethacrylate (PEG2k-DMA), and 1,2-distearyloxypropyl-3-amine-N-[methoxy (polyethylene glycol)-2000] (PEG2k-DSA). In one particular example, the stealth lipid may be PEG2k-DMG.

The LNPs can comprise different respective molar ratios of the component lipids in the formulation. The mol-% of the CCD lipid may be, for example, from about 30 mol-% to about 60 mol-%, from about 35 mol-% to about 55 mol-%, from about 40 mol-% to about 50 mol-%, from about 42 mol-% to about 47 mol-%, or about 45%. The mol-% of the helper lipid may be, for example, from about 30 mol-% to about 60 mol-%, from about 35 mol-% to about 55 mol-%, from about 40 mol-% to about 50 mol-%, from about 41 mol-% to about 46 mol-%, or about 44 mol-%. The mol-% of the neutral lipid may be, for example, from about 1 mol-% to about 20 mol-%, from about 5 mol-% to about 15 mol-%, from about 7 mol-% to about 12 mol-%, or about 9 mol-%. The mol-% of the stealth lipid may be, for example, from about 1 mol-% to about 10 mol-%, from about 1 mol-% to about 5 mol-%, from about 1 mol-% to about 3 mol-%, about 2 mol-%, or about 1 mol-%.

The LNPs can have different ratios between the positively charged amine groups of the biodegradable lipid (N) and the negatively charged phosphate groups (P) of the nucleic acid to be encapsulated. This may be mathematically represented by the equation N/P. For example, the N/P ratio may be from about 0.5 to about 100, from about 1 to about 50, from about 1 to about 25, from about 1 to about 10, from about 1 to about 7, from about 3 to about 5, from about 4 to about 5, about 4, about 4.5, or about 5. The N/P ratio can also be from about 4 to about 7 or from about 4.5 to about 6. In specific examples, the N/P ratio can be 4.5 or can be 6.

A specific example of a suitable LNP has a nitrogen-to-phosphate (N/P) ratio of 4.5 and contains biodegradable cationic lipid, cholesterol, DSPC, and PEG2k-DMG in a 45:44:9:2 molar ratio. The biodegradable cationic lipid can be (9Z,12Z)-3-((4,4-bis(octyloxy) butanoyl)oxy)-2-((((3-(diethylamino) propoxy-) carbonyl)oxy)methyl) propyl octadeca-9,12-dienoate, also called 3-((4,4-bis(octyloxy) butanoyl)oxy)-2-((((3-(diethylamino) propoxy) carbonyl-)oxy)methyl) propyl (9Z,12Z)-octadeca-9,12-dienoate. See, e.g., Finn et al. (2018) Cell Rep. 22 (9): 2227-2235, herein incorporated by reference in its entirety for all purposes. Another specific example of a suitable LNP contains Dlin-MC3-DMA (MC3), cholesterol, DSPC, and PEG-DMG in a 50:38.5:10:1.5 molar ratio.

Another specific example of a suitable LNP has a nitrogen-to-phosphate (N/P) ratio of 6 and contains biodegradable cationic lipid, cholesterol, DSPC, and PEG2k-DMG in a 50:38:9:3 molar ratio. The biodegradable cationic lipid can be (9Z,12Z)-3-((4,4-bis(octyloxy) butanoyl)oxy)-2-((((3-(diethylamino) propoxy-) carbonyl)oxy)methyl) propyl octadeca-9,12-dienoate, also called 3-((4,4-bis(octyloxy) butanoyl)oxy)-2-((((3-(diethylamino) propoxy) carbonyl-)oxy) methyl) propyl (9Z,12Z)-octadeca-9,12-dienoate.

Another specific example of a suitable LNP has a nitrogen-to-phosphate (N/P) ratio of 3 and contains a cationic lipid, a structural lipid, cholesterol (e.g., cholesterol (ovine) (Avanti 700000)), and PEG2k-DMG (e.g., PEG-DMG 2000 (NOF America-STJNBRIGHT® GM-020 (DMG-PEG)) in a 50:10:38.5:1.5 ratio or a 47:10:42:1 ratio. The structural lipid can be, for example, DSPC (e.g., DSPC (Avanti 850365)), SOPC, DOPC, or DOPE. The cationic/ionizable lipid can be, for example, Dlin-MC3-DMA (e.g., Dlin-MC3-DMA (Biofine International)).

Another specific example of a suitable LNP contains Dlin-MC3-DMA, DSPC, cholesterol, and a PEG lipid in a 45:9:44:2 ratio. Another specific example of a suitable LNP contains Dlin-MC3-DMA, DOPE, cholesterol, and PEG lipid or PEG DMG in a 50:10:39:1 ratio. Another specific example of a suitable LNP has Dlin-MC3-DMA, DSPC, cholesterol, and PEG2k-DMG at a 55:10:32.5:2.5 ratio. Another specific example of a suitable LNP has Dlin-MC3-DMA, DSPC, cholesterol, and PEG-DMG in a 50:10:38.5:1.5 ratio. Another specific example of a suitable LNP has Dlin-MC3-DMA, DSPC, cholesterol, and PEG-DMG in a 50:10:38.5:1.5 ratio.

When administered to a subject, the effector molecule is cleaved, and concentrated (retained) bioactive effector (e.g. IL-12) is released within the TME.

The present disclosure also provides a method of treating a disease in a subject in need thereof, the method comprising administering a therapeutically effective amount of the pharmaceutical composition of the disclosure. In certain embodiments, the disease is cancer. The pharmaceutical composition may be administered intra-tumorally, locally in the TME, or systemically.

### EXAMPLES

The mRNA constructs encoding for membrane-bound IL-12p70 and membrane-bound IL-12p70 harboring protease cleavable linkers were validated utilizing a Chinese Hamster Ovarian (CHO) transfection system. The CHO transfection system was treated with either vehicle control, or a matrix metalloprotease 2 (MMP2) to detect cleavage of IL-12p70 from its membrane-linked localization into its soluble form. Once validated, the anti-tumor efficacy of local delivery of lipid nanoparticles (LNP) encapsulating these mRNA constructs encoding for various IL-12p70 sequences (soluble, membrane-linked, membrane-linked with protease cleavable substrates) was determined. For these syngeneic tumor growth studies, we utilized two relevant in vivo models (B16F10 melanoma, MC38 colorectal cancer) where tumor cells were subcutaneously injected into the flank of the C57BL/6 strain of mice purchased from Jackson Laboratories. For the in vivo studies, mice were divided evenly according to the tumor size (measured by caliper with the calculation formula: (L²×W)/2 where L is the smallest size).

TABLE 1
Non-cleavable or protease-cleavable linker
amino acid sequences utilized for in vitro
Study 1, in vivo Study 2 and in vivo Study 3.

| Linker Name | Protein Sequence |
|---|---|
| TM (SEQ ID NO: 1) | GGGGSGGGGSGGGGS |
| L1 (SEQ ID NO: 2) | GGGGSGGGGSGGGGSGGGS |
| L2 (SEQ ID NO: 3) | GGGGSISSGLLSGRSDNHGGGGS |
| L3 (SEQ ID NO: 4) | GGGGSVPLSLYSGGGISSGLLSGR |
| | SDNHGGGGS |
| L4 (SEQ ID NO: 5) | GGGGSHPVGLLARGGGHPVGLLAR |
| | GGSGRSAGGSGRSAGGGGS |
| L4s (SEQ ID NO: 6) | GGGGSHPVGLLARGGGGS |
| L5 (SEQ ID NO: 7) | GGGGSLAQAVRSSGGGGS |

### Example 1. In Vitro Validation of mRNA Constructs

In vitro Study 1 aimed to validate the various mRNA constructs described in Table 1. This study involved CHO cell transfection where specific wells were transfected with 500 ng of mRNA encoding for F-Luciferase (negative control), membrane-linked IL-12p70 (TM, L1) or membrane-linked IL-12p70 with protease cleavable substrates (L2, L3, L4s, L5) as indicated in Table 1 where the bolded sequences are protease-binding substrates. 16 hours post-transfection, these corresponding wells were treated with phosphate-buffered saline (PBS) or 1 μg/mL of the activated protease, MMP2. MMP2 was activated by pre-treating the recombinant MMP2 with 4-aminophenylmercuric acetate for 1 hour at 37° C. Cells were treated for 180 minutes and then subsequently gently pipetted to detach and stain for cell surface expression of IL-12 with an antibody detecting IL-12p40 conjugated to PerCP-Cy5.5 from Biolegend (Cat. #505212, clone C15.6). Enzymatic detachment was avoided to prevent interference/degradation of surface protein expression. Samples were acquired on a Becton Dickinson LSR Fortessa Flow Cytometer. Similarly, supernatants, including soluble IL-12p70 (positive control) or mCherry (negative control) were harvested at 90 minutes and 180 minutes and were subjected to ELISA quantikine detection of IL-12p70.

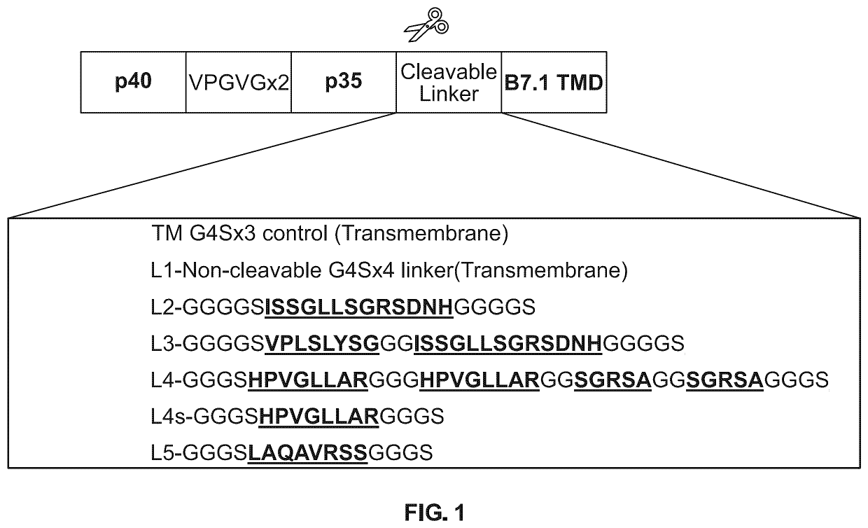

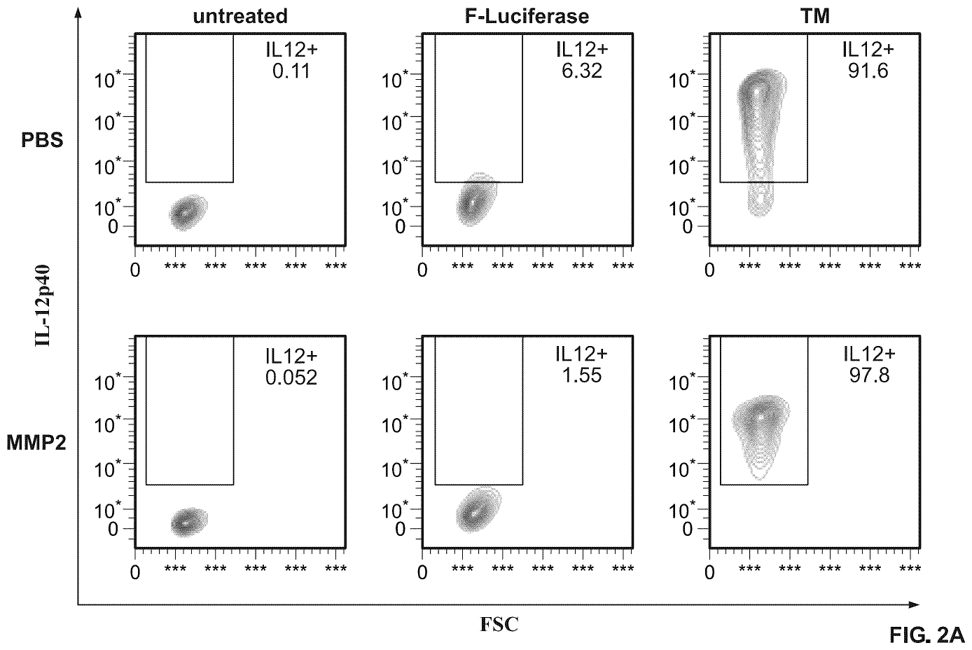

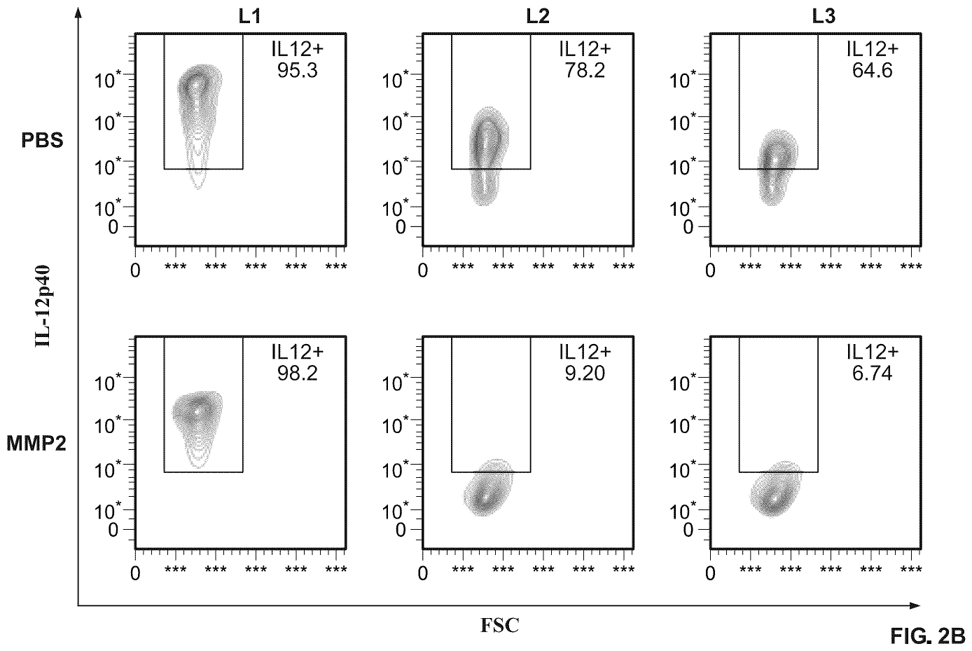

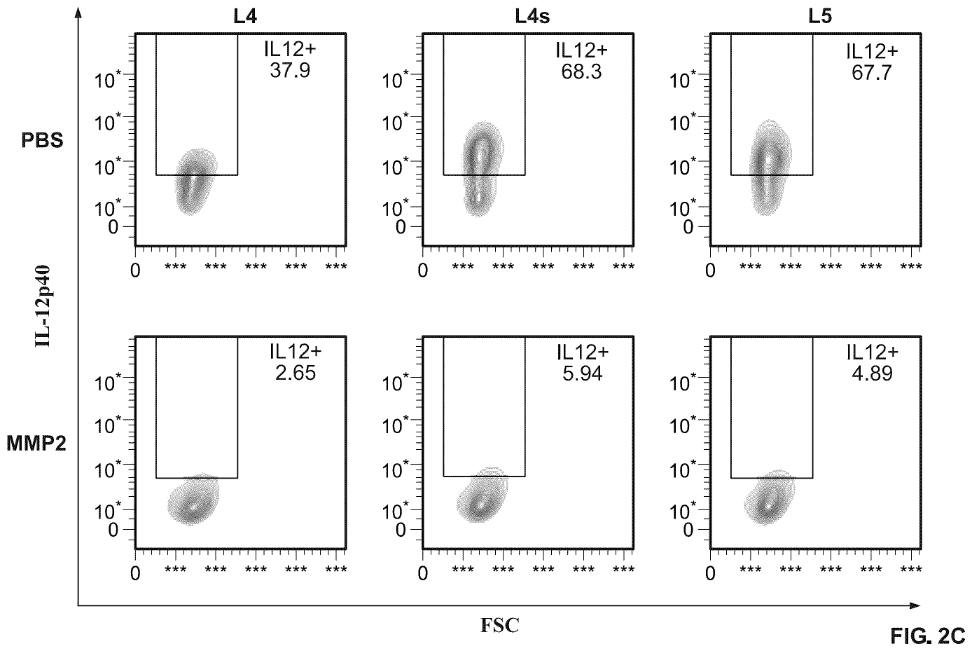

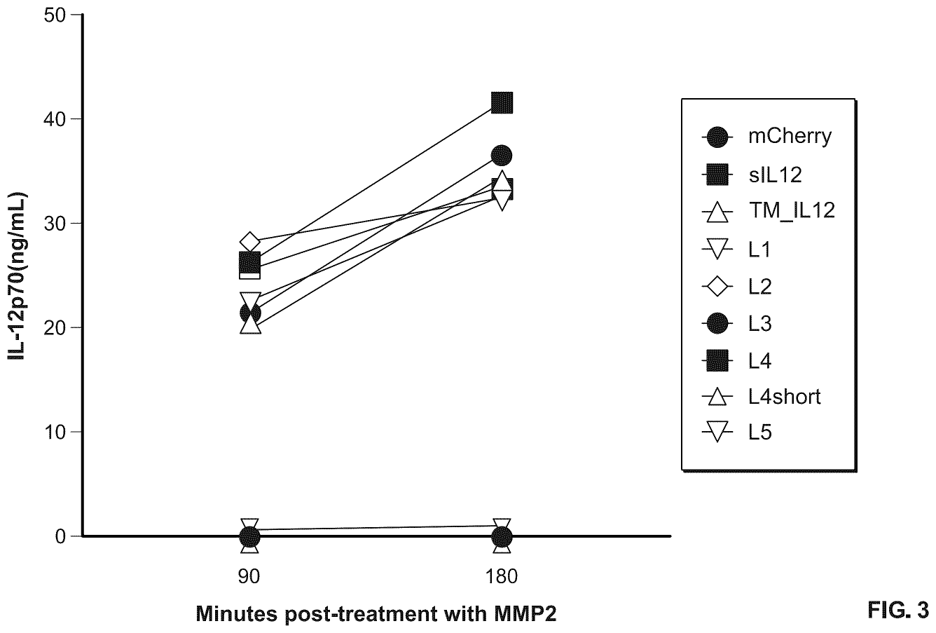

This study showed that mRNA constructs encoding for IL-12p70-TM sequences with different protease cleavable linker substrate(s) resulted in cleavage upon MMP2 treatment. This example describes the various IL-12p70 transmembrane-bound or IL-12p70 transmembrane-bound linked to protease cleavable substrate mRNA sequences utilized in this report (FIG. 1). Using a Chinese hamster ovarian cell (CHO) mRNA transfection system, these constructs were validated in two different assays using exogenous addition of MMP2, a potent protease with broad specificity. Using flow cytometric analysis following 180 minutes of MMP2 treatment, the transmembrane (TM and L1) sequences showed no impact on surface IL-12p40 staining. However, sequences harboring cleavable linker substrates (L2, L3, L4, L4s, L5) all showed pronounced reductions in cell-surface staining of IL-12p40 (FIGS. 2A-2C) suggesting efficient cleavage. In parallel, utilizing an enzyme-linked immunosorbent assay (ELISA) to detect release of cleaved (therefore soluble) IL-12p70, a time-dependent release of IL-12p70 was observed confirming expression and validity of the protease-cleavable linker sequences within each construct (FIG. 3).

### Example 2. In Vivo Study of Anti-Tumor Activity of Chimeric Proteins in Mice Injected with B16F10 Melanoma Tumor Cells

In vivo Study 2 involved nine groups with seven to eight mice per group. At day 0, mice were anesthetized by isoflurane inhalation and then injected subcutaneously into the right flank with 3.5×10⁵ B16F10 melanoma tumor cells in suspension of 50 μL of medium. At day 10, mice were randomized evenly within the nine groups according to the tumor sizes to average 100 mm³, and mice received an intratumoral injection of 10 μL of LNP-mRNA in in PBS encoding for F-luciferase, soluble IL-12p70 (sIL-12), membrane-linked IL-12p70 (TM, L1) or membrane-linked IL-12p70 with protease cleavable substrates (L1, L2, L3, L4s, L5). Mice were treated every 3 days for a total of four intratumoral LNP-mRNA injections. LNPs utilizing fixed concentrations of ionizable lipids, cholesterol, structural lipids and polyethylene glycol (PEG) were used. Experimental dosing and treatment protocol for groups of mice are shown in Table 2.

Intratumoral delivery of LNP-mRNA constructs encoding for IL-12p70-membrane bound including protease cleavable linker substrate(s) sequences resulted in greater anti-tumor efficacy than IL-12p70-membrane bound stable form in the B16F10 tumor model. This example describes the potent anti-tumor efficacy of intratumoral delivery of LNP-mRNA encoding for membrane-bound IL-12p70 harboring protease-cleavable linker sequences in the aggressive B16F10 melanoma tumor model. Soluble IL-12p70 mRNA, which has shown robust anti-tumor efficacy in previous studies, was used as a benchmark to compare among anti-tumor efficacy of the tested constructs. Similarly, membrane-bound IL-12p70 showed modest efficacy in previous studies and was also used to benchmark anti-tumor efficacy of the tested constructs.

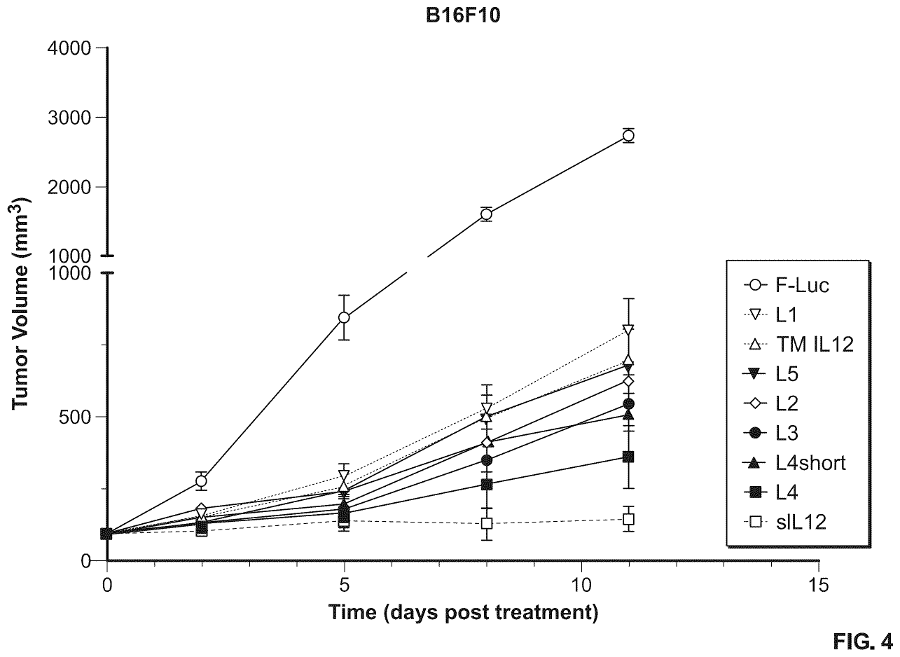

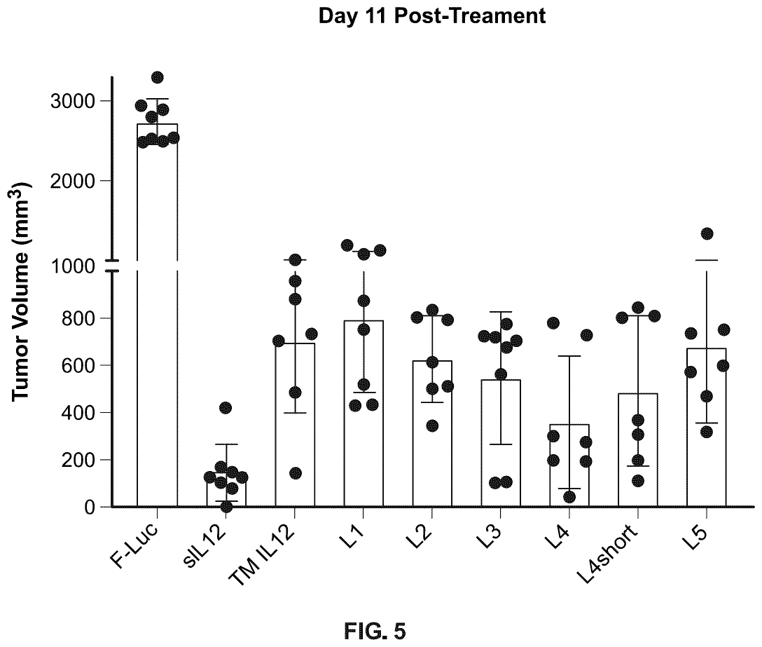

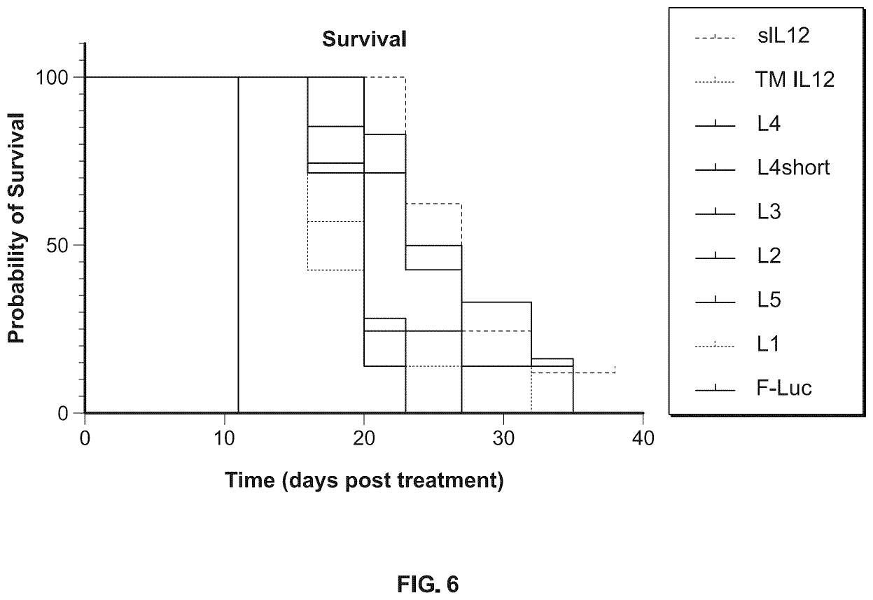

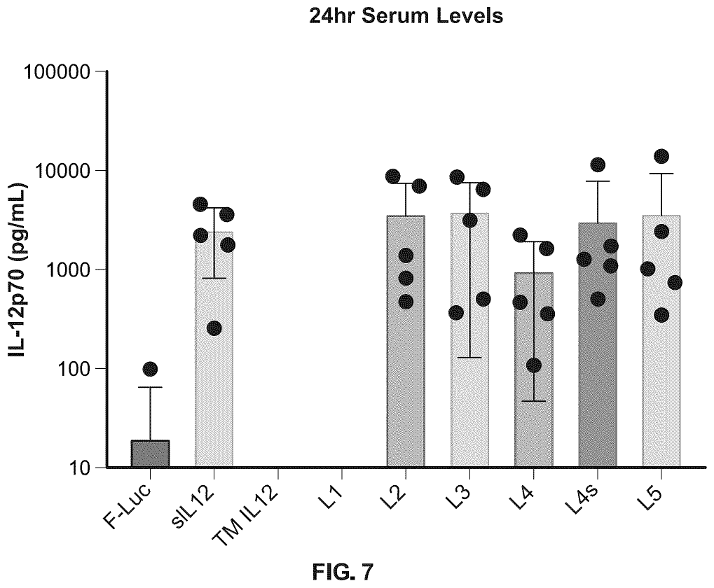

C57BL/6 strain background mice from Jackson Laboratories were implanted subcutaneously with B16F10 cells (3.5×10⁵ cells/mouse) at day 0. Tumors were measured using a caliper and tumor volumes were calculated with the formula (L²×W)/2 where L is the smallest size. Mice were randomized evenly into nine treatment groups when the average tumor size reached 100 mm³ which was at day 10. Mice received an intratumoral injection of 10 μg of LNP-mRNA on day 10, day 13, day 16 and day 19 encoding for F-Luciferase as a control, soluble IL-12p70, membrane-bound IL-12p70 (IL-12p70_TM) or the various sequences found in FIG. 1 describing linker-length controls (L1) or linkers between IL-12p70 and the transmembrane signal peptide that harbor protease-substrate sequences for cleavage into soluble IL-12p70. Tumor volumes were monitored by caliper measurements twice per week until the end of the study at day 45. The average of tumor volumes over time for each group shows that all treatment groups resulted in strong anti-tumor efficacy as compared to the F-luciferase control. As expected, soluble IL-12p70 had the greatest overall anti-tumor efficacy and survival (FIGS. 4, 5 and 6). Interestingly, most sequences harboring protease-cleavable linkers had stronger efficacy as compared to the membrane-linked sequences, TM and L1 (FIGS. 4, 5 and 6). Specifically, Linkers L4, L4-short and L3 had the strongest overall efficacy. Furthermore, serum was collected from animals 24 hours post-initial dosing and revealed that while no circulating IL-12p70 was found within the transmembrane-linked sequences, all protease-cleavable sequences resulted in elevated levels of circulating IL-12p70 suggesting these proteins are indeed cleaved in vivo (FIG. 7).

TABLE 2
Mean tumor volume and standard deviation for treatment groups
for in vivo Study 1 using the B16F10 melanoma tumor model.

| | | Tumor | Tumor |
|---|---|---|---|
| | | Volume, mm³ | Volume, mm³ |
| | | mean (±SD) | mean (±SD) |
| Treatment group (n = 7-8) | | Day 18 | Day 21 |
| 1 | F-Luciferase | 1591 (±255) | 2741 (±286) |
| 2 | sIL-12 | 131 (±162) | 146 (±122) |
| 3 | TM | 493 (±229) | 702 (±304) |
| 4 | L1 | 534 (±225) | 802 (±317) |
| 5 | L2 | 490 (±130) | 628 (±187) |
| 6 | L3 | 348 (±192) | 547 (±280) |
| 7 | L4 | 266 (±227) | 360 (±283) |
| 8 | L4s | 411 (±254) | 491 (±318) |
| 9 | L5 | 503 (±127) | 684 (±324) |

As shown in Table 2, for in vivo Study 1 using B16F10 subcutaneous tumor model, mice treated with sIL-12p70 had robust anti-tumor efficacy. Interestingly, all constructs tested harboring protease-cleavable linkers between IL-12p70 and the transmembrane domain (L2, L3, L4, L4s, L5) had better anti-tumor efficacy compared to the modest efficacy seen with membrane-bound IL-12p70 (TM, L1).

### Example 3. In Vivo Study of Anti-Tumor Activity of Chimeric Proteins in Mice Injected with MC38 Colorectal Tumor Cells

In vivo Study 3 involved nine groups with six to seven mice per group. At day 0, mice were anesthetized by isoflurane inhalation and then injected subcutaneously into the right flank with 3×10⁵ MC38 colorectal tumor cells in suspension of 50 μL of media. At day 11, mice were randomized evenly within the nine groups according to the tumor sizes to average 85 mm³, and mice received an intratumoral injection of 10 μL of LNP-mRNA in PBS encoding for F-luciferase, soluble IL-12p70 (sIL12), membrane-linked IL-12p70 (TM, L1) or membrane-linked IL-12p70 with protease cleavable substrates (L1, L2, L3, L4s, L5). Mice were treated every 3 days for a total of four LNP-mRNA intratumoral injections. LNPs utilizing fixed concentrations of ionizable lipids, cholesterol, structural lipids and polyethylene glycol (PEG) were used. Experimental dosing and treatment protocol for groups of mice are shown in Table 3.

Intratumoral delivery of LNP-mRNA constructs encoding for IL-12p70-membrane bound including protease linker substrate(s) sequences resulted in greater anti-tumor efficacy than IL-12p70-membrane bound stable form in the MC38 tumor model. This example describes the potent anti-tumor efficacy of intratumoral delivery of LNP-mRNA encoding for membrane-bound IL-12p70 harboring protease-cleavable linker sequences in the aggressive MC38 colorectal tumor model. Soluble IL-12p70 mRNA, which has shown robust anti-tumor efficacy in previous studies was used as a benchmark to compare among anti-tumor efficacy of the tested constructs. Similarly, membrane-bound IL-12p70 showed modest efficacy in previous studies and was also used to benchmark anti-tumor efficacy of the tested constructs.

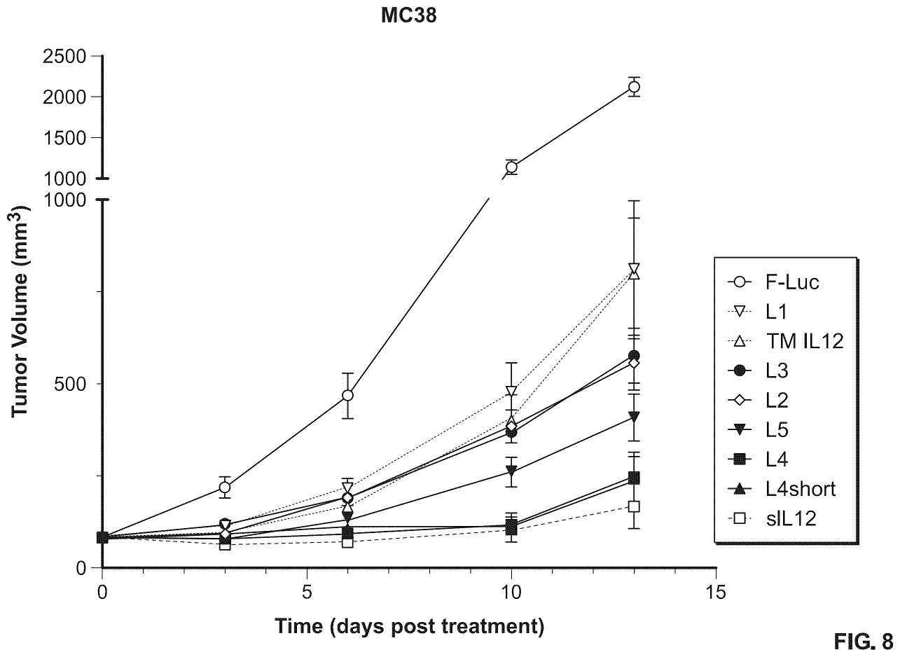

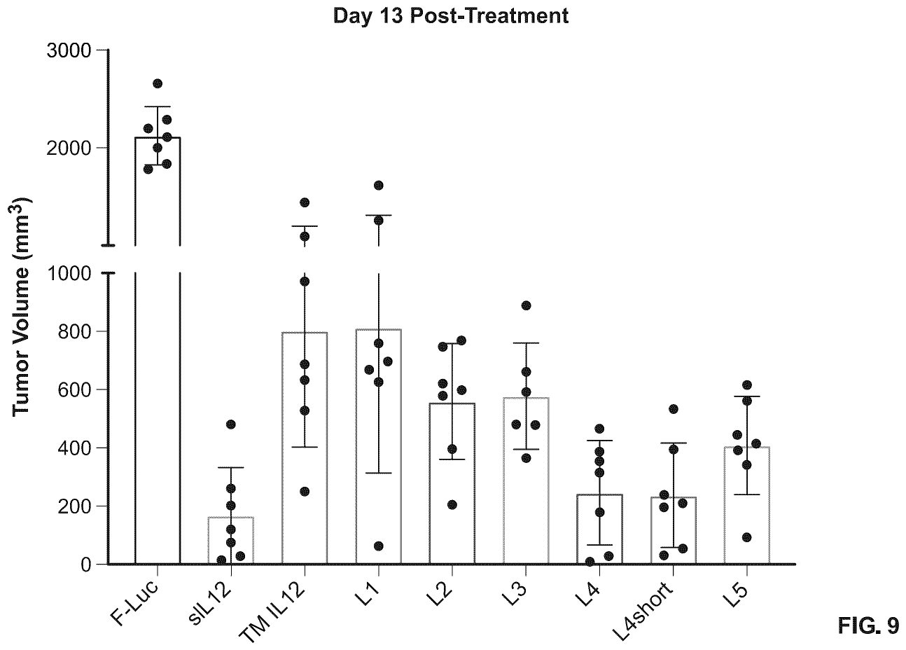

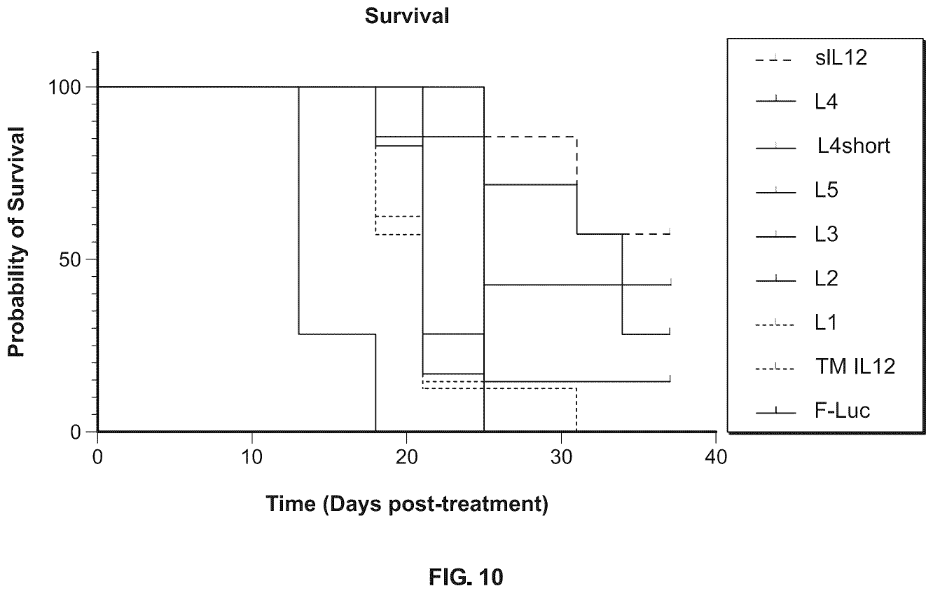

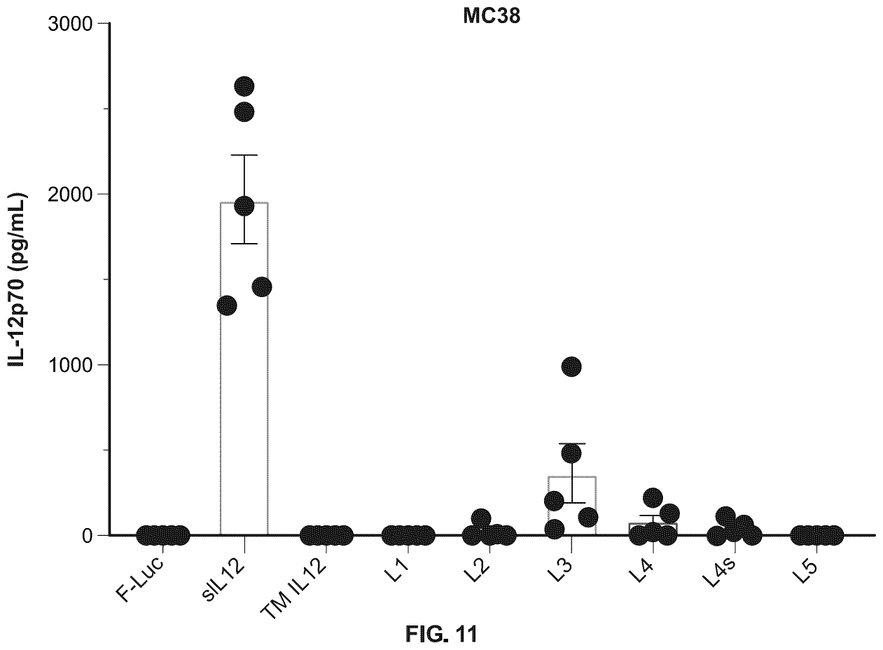

C57BL/6 strain background mice from Jackson Laboratories were implanted subcutaneously with MC38 cells (3×10⁵ cells/mouse) at day 0. Tumors were measured using a caliper and tumor volumes were calculated with the formula (L²×W)/2 where L is the smallest size. Mice were randomized evenly into nine treatment groups when the average tumor size reached 85 mm³ which was at day 11. Mice received an intratumoral injection of 10 μg of LNP-mRNA on day 11, day 14, day 17 and day 20 encoding for F-Luciferase as a control, soluble IL-12p70, membrane-bound IL-12p70 or the various sequences found in FIG. 1 describing linker-length controls (L1) or linkers between IL-12p70 and the transmembrane signal peptide that harbor protease-substrate sequences for cleavage into soluble IL-12p70. Tumor volumes were monitored by caliper measurements twice per week until the end of the study at day 45. Similarly to in vivo study 1, soluble IL-12p70 had the greatest overall anti-tumor efficacy and survival (FIGS. 8, 9 and 10). Interestingly, most sequences harboring protease-cleavable linkers had stronger efficacy as compared to the membrane-linked sequences, TM and L1 (FIGS. 8, 9 and 10). Specifically, linkers L4-short, L4, L5 and L2 had the best overall trend in anti-tumor efficacy (in that order) and resulted in 3, 2, 1 and 1 tumor-free mice, respectively. Furthermore, serum was collected from animals 24 hours post-initial dosing and revealed that while no circulating IL-12p70 was found within the membrane-linked sequences, all protease-cleavable sequences resulted in elevated levels of circulating IL-12p70 suggesting these proteins are indeed cleaved in vivo (FIG. 11).

TABLE 3
Mean tumor volume and standard deviation for treatment groups
for in vivo Study 2 using the MC38 colorectal tumor model.

| | | Tumor | Tumor | |
|---|---|---|---|---|
| | | Volume, mm³ | Volume, mm³ | Survival |
| | | mean (±SD) | mean (±SD) | (%) |
| Treatment group (n = 6-7) | | Day 21 | Day 24 | D 60 |
| 1 | F-Luciferase | 1140 (±223) | 2127 (±300) | 0 |
| 2 | sIL-12 | 105 (±90) | 167 (±163) | 57.1 |
| 3 | TM | 407 (±173) | 801 (±398) | 0 |
| 4 | L1 | 479 (±213) | 811 (±496) | 0 |
| 5 | L2 | 384 (±119) | 558 (±198) | 14.3 |
| 6 | L3 | 369 (±76) | 577 (±182) | 0 |
| 7 | LA | 121 (±81) | 247 (±179) | 28.6 |
| 8 | L4s | 114 (±63) | 236 (±178) | 42.9 |
| 9 | L5 | 261 (±109) | 408 (±169) | 14.3 |

As shown in Table 3, for in vivo Study 2 using the MC38 subcutaneous tumor model, mice treated with sIL-12p70 had robust anti-tumor efficacy. Interestingly, all constructs tested harboring protease-cleavable linkers between IL-12p70 and the transmembrane domain (L2, L3, L4, L4s, L5) had better anti-tumor efficacy compared to the modest efficacy seen with membrane-bound IL-12p70 (TM, L1).

The present disclosure has been described in detail, including the preferred embodiments thereof. However, it will be appreciated that those skilled in the art, upon consideration of the present disclosure, may make modifications and/or improvements on this disclosure that fall within the scope and spirit of the disclosure.

Sequences of IL-12p70 constructs with or without
cleavable peptide linkers
IL-12p70
(SEQ ID NO: 17)
ATGTGTCCTCAGAAGCTAACCATCTCCTGGTTTGCCATCGTTTTGCTGGT
GTCTCCACTCATGGCCATGTGGGAGCTGGAGAAAGACGTTTATGTTGTAG
AGGTGGACTGGACTCCCGATGCCCCTGGAGAAACAGTGAACCTCACCTGT
GACACGCCTGAAGAAGATGACATCACCTGGACCTCAGACCAGAGACATGG
AGTCATAGGCTCTGGAAAGACCCTGACCATCACTGTCAAAGAGTTTCTAG
ATGCTGGCCAGTACACCTGCCACAAAGGAGGCGAGACTCTGAGCCACTCA
CATCTGCTGCTCCACAAGAAGGAAAATGGAATTTGGTCCACTGAAATTTT
AAAAAATTTCAAAAACAAGACTTTCCTGAAGTGTGAAGCACCAAATTACT
CCGGACGGTTCACGTGCTCATGGCTGGTGCAAAGAAACATGGACTTGAAG
TTCAACATCAAGAGCAGTAGCAGTTCCCCTGACTCTCGGGCAGTGACATG
TGGAATGGCGTCTCTGTCTGCAGAGAAGGTCACACTGGACCAAAGGGACT
ATGAGAAGTATTCAGTGTCCTGCCAGGAGGATGTCACCTGCCCAACTGCC
GAGGAGACCCTGCCCATTGAACTGGCGTTGGAAGCACGGCAGCAGAATAA
ATATGAGAACTACAGCACCAGCTTCTTCATCAGGGACATCATCAAACCAG
ACCCGCCCAAGAACTTGCAGATGAAGCCTTTGAAGAACTCACAGGTGGAG
GTCAGCTGGGAGTACCCTGACTCCTGGAGCACTCCCCATTCCTACTTCTC
CCTCAAGTTCTTTGTTCGAATCCAGCGCAAGAAAGAAAAGATGAAGGAGA
CAGAGGAGGGGTGTAACCAGAAAGGTGCGTTCCTCGTAGAGAAGACATCT
ACCGAAGTCCAATGCAAAGGCGGGAATGTCTGCGTGCAAGCTCAGGATCG
CTATTACAATTCCTCATGCAGCAAGTGGGCATGTGTTCCCTGCAGAGTCC
GATCGGTTCCTGGAGTAGGGGTACCTGGAGTGGGCAGGGTCATACCGGTC
TCTGGACCTGCCAGGTGTCTTAGCCAGTCCCGAAACCTGCTGAAGACCAC
AGATGACATGGTGAAGACGGCCAGAGAAAAGCTGAAACATTATTCCTGCA
CTGCTGAAGACATCGATCATGAAGACATCACACGGGACCAAACCAGCACA
TTGAAGACCTGTTTACCACTGGAACTACACAAGAACGAGAGTTGCCTGGC
TACTAGAGAGACTTCTTCCACAACAAGAGGGAGCTGCCTGCCCCCACAGA
AGACGTCTTTGATGATGACCCTGTGCCTTGGTAGCATCTATGAGGACTTG
AAGATGTACCAGACAGAGTTCCAGGCCATCAACGCAGCACTTCAGAATCA
CAACCATCAGCAGATCATTCTAGACAAGGGCATGCTGGTGGCCATCGATG
AGCTGATGCAGTCTCTGAATCATAATGGCGAGACTCTGCGCCAGAAACCT
CCTGTGGGAGAAGCAGACCCTTACAGAGTGAAAATGAAGCTCTGCATCCT
GCTTCACGCCTTCAGCACCCGCGTCGTGACCATCAACAGGGTGATGGGCT
ATCTGAGCTCCGCCTGA
IL-12p70 TM (membrane bound)
(SEQ ID NO: 18)
ATGTGTCCTCAGAAGCTAACCATCTCCTGGTTTGCCATCGTTTTGCTGGT
GTCTCCACTCATGGCCATGTGGGAGCTGGAGAAAGACGTTTATGTTGTAG
AGGTGGACTGGACTCCCGATGCCCCTGGAGAAACAGTGAACCTCACCTGT
GACACGCCTGAAGAAGATGACATCACCTGGACCTCAGACCAGAGACATGG
AGTCATAGGCTCTGGAAAGACCCTGACCATCACTGTCAAAGAGTTTCTAG
ATGCTGGCCAGTACACCTGCCACAAAGGAGGCGAGACTCTGAGCCACTCA
CATCTGCTGCTCCACAAGAAGGAAAATGGAATTTGGTCCACTGAAATTTT
AAAAAATTTCAAAAACAAGACTTTCCTGAAGTGTGAAGCACCAAATTACT
CCGGACGGTTCACGTGCTCATGGCTGGTGCAAAGAAACATGGACTTGAAG
TTCAACATCAAGAGCAGTAGCAGTTCCCCTGACTCTCGGGCAGTGACATG
TGGAATGGCGTCTCTGTCTGCAGAGAAGGTCACACTGGACCAAAGGGACT
ATGAGAAGTATTCAGTGTCCTGCCAGGAGGATGTCACCTGCCCAACTGCC
GAGGAGACCCTGCCCATTGAACTGGCGTTGGAAGCACGGCAGCAGAATAA
ATATGAGAACTACAGCACCAGCTTCTTCATCAGGGACATCATCAAACCAG
ACCCGCCCAAGAACTTGCAGATGAAGCCTTTGAAGAACTCACAGGTGGAG
GTCAGCTGGGAGTACCCTGACTCCTGGAGCACTCCCCATTCCTACTTCTC
CCTCAAGTTCTTTGTTCGAATCCAGCGCAAGAAAGAAAAGATGAAGGAGA
CAGAGGAGGGGTGTAACCAGAAAGGTGCGTTCCTCGTAGAGAAGACATCT
ACCGAAGTCCAATGCAAAGGCGGGAATGTCTGCGTGCAAGCTCAGGATCG
CTATTACAATTCCTCATGCAGCAAGTGGGCATGTGTTCCCTGCAGAGTCC
GATCGGTTCCTGGAGTAGGGGTACCTGGAGTGGGCAGGGTCATACCGGTC
TCTGGACCTGCCAGGTGTCTTAGCCAGTCCCGAAACCTGCTGAAGACCAC
AGATGACATGGTGAAGACGGCCAGAGAAAAGCTGAAACATTATTCCTGCA
CTGCTGAAGACATCGATCATGAAGACATCACACGGGACCAAACCAGCACA
TTGAAGACCTGTTTACCACTGGAACTACACAAGAACGAGAGTTGCCTGGC
TACTAGAGAGACTTCTTCCACAACAAGAGGGAGCTGCCTGCCCCCACAGA
AGACGTCTTTGATGATGACCCTGTGCCTTGGTAGCATCTATGAGGACTTG
AAGATGTACCAGACAGAGTTCCAGGCCATCAACGCAGCACTTCAGAATCA
CAACCATCAGCAGATCATTCTAGACAAGGGCATGCTGGTGGCCATCGATG
AGCTGATGCAGTCTCTGAATCATAATGGCGAGACTCTGCGCCAGAAACCT
CCTGTGGGAGAAGCAGACCCTTACAGAGTGAAAATGAAGCTCTGCATCCT
GCTTCACGCCTTCAGCACCCGCGTCGTGACCATCAACAGGGTGATGGGCT
ATCTGAGCTCCGCCGGGGGGGGAGGCAGTGGAGGCGGTGGTTCAGGTGGT
GGTGGGTCCAACACACTTGTGCTCTTTGGGGCAGGATTCGGCGCAGTAAT
AACAGTCGTCGTCATCGTTGTCATCATCAAATGCTTCTGTAAGCACAGAA
GCTGTTTCAGAAGAAGAAATGAGGCAAGCAGAGAAACAAACAACAGCCTT
ACCTTCGGGCCTGAAGAAGCATTAGCTGAACAGACCGTCTTCCTTTAG
IL-12p70-L1-TM
(SEQ ID NO: 19)
ATGTGTCCTCAGAAGCTAACCATCTCCTGGTTTGCCATCGTTTTGCTGGT
GTCTCCACTCATGGCCATGTGGGAGCTGGAGAAAGACGTTTATGTTGTAG
AGGTGGACTGGACTCCCGATGCCCCTGGAGAAACAGTGAACCTCACCTGT
GACACGCCTGAAGAAGATGACATCACCTGGACCTCAGACCAGAGACATGG
AGTCATAGGCTCTGGAAAGACCCTGACCATCACTGTCAAAGAGTTTCTAG
ATGCTGGCCAGTACACCTGCCACAAAGGAGGCGAGACTCTGAGCCACTCA
CATCTGCTGCTCCACAAGAAGGAAAATGGAATTTGGTCCACTGAAATTTT
AAAAAATTTCAAAAACAAGACTTTCCTGAAGTGTGAAGCACCAAATTACT
CCGGACGGTTCACGTGCTCATGGCTGGTGCAAAGAAACATGGACTTGAAG
TTCAACATCAAGAGCAGTAGCAGTTCCCCTGACTCTCGGGCAGTGACATG
TGGAATGGCGTCTCTGTCTGCAGAGAAGGTCACACTGGACCAAAGGGACT
ATGAGAAGTATTCAGTGTCCTGCCAGGAGGATGTCACCTGCCCAACTGCC
GAGGAGACCCTGCCCATTGAACTGGCGTTGGAAGCACGGCAGCAGAATAA
ATATGAGAACTACAGCACCAGCTTCTTCATCAGGGACATCATCAAACCAG
ACCCGCCCAAGAACTTGCAGATGAAGCCTTTGAAGAACTCACAGGTGGAG
GTCAGCTGGGAGTACCCTGACTCCTGGAGCACTCCCCATTCCTACTTCTC
CCTCAAGTTCTTTGTTCGAATCCAGCGCAAGAAAGAAAAGATGAAGGAGA
CAGAGGAGGGGTGTAACCAGAAAGGTGCGTTCCTCGTAGAGAAGACATCT
ACCGAAGTCCAATGCAAAGGCGGGAATGTCTGCGTGCAAGCTCAGGATCG
CTATTACAATTCCTCATGCAGCAAGTGGGCATGTGTTCCCTGCAGAGTCC
GATCGGTTCCTGGAGTAGGGGTACCTGGAGTGGGCAGGGTCATACCGGTC
TCTGGACCTGCCAGGTGTCTTAGCCAGTCCCGAAACCTGCTGAAGACCAC
AGATGACATGGTGAAGACGGCCAGAGAAAAGCTGAAACATTATTCCTGCA
CTGCTGAAGACATCGATCATGAAGACATCACACGGGACCAAACCAGCACA
TTGAAGACCTGTTTACCACTGGAACTACACAAGAACGAGAGTTGCCTGGC
TACTAGAGAGACTTCTTCCACAACAAGAGGGAGCTGCCTGCCCCCACAGA
AGACGTCTTTGATGATGACCCTGTGCCTTGGTAGCATCTATGAGGACTTG
AAGATGTACCAGACAGAGTTCCAGGCCATCAACGCAGCACTTCAGAATCA
CAACCATCAGCAGATCATTCTAGACAAGGGCATGCTGGTGGCCATCGATG
AGCTGATGCAGTCTCTGAATCATAATGGCGAGACTCTGCGCCAGAAACCT
CCTGTGGGAGAAGCAGACCCTTACAGAGTGAAAATGAAGCTCTGCATCCT
GCTTCACGCCTTCAGCACCCGCGTCGTGACCATCAACAGGGTGATGGGCT
ATCTGAGCTCCGCCGGGGGGGGAGGCAGTGGAGGCGGTGGTTCAGGTGGT
GGTGGGTCCAACACACTTGTGCTCTTTGGGGCAGGATTCGGCGCAGTAAT
AACAGTCGTCGTCATCGTTGTCATCATCAAATGCTTCTGTAAGCACAGAA
GCTGTTTCAGAAGAAGAAATGAGGCAAGCAGAGAAACAAACAACAGCCTT
ACCTTCGGGCCTGAAGAAGCATTAGCTGAACAGACCGTCTTCCTTTAG
IL-12p70-L2-TM
(SEQ ID NO: 20)
ATGTGTCCTCAGAAGCTAACCATCTCCTGGTTTGCCATCGTTTTGCTGGT
GTCTCCACTCATGGCCATGTGGGAGCTGGAGAAAGACGTTTATGTTGTAG
AGGTGGACTGGACTCCCGATGCCCCTGGAGAAACAGTGAACCTCACCTGT
GACACGCCTGAAGAAGATGACATCACCTGGACCTCAGACCAGAGACATGG
AGTCATAGGCTCTGGAAAGACCCTGACCATCACTGTCAAAGAGTTTCTAG
ATGCTGGCCAGTACACCTGCCACAAAGGAGGCGAGACTCTGAGCCACTCA
CATCTGCTGCTCCACAAGAAGGAAAATGGAATTTGGTCCACTGAAATTTT
AAAAAATTTCAAAAACAAGACTTTCCTGAAGTGTGAAGCACCAAATTACT
CCGGACGGTTCACGTGCTCATGGCTGGTGCAAAGAAACATGGACTTGAAG
TTCAACATCAAGAGCAGTAGCAGTTCCCCTGACTCTCGGGCAGTGACATG
TGGAATGGCGTCTCTGTCTGCAGAGAAGGTCACACTGGACCAAAGGGACT
ATGAGAAGTATTCAGTGTCCTGCCAGGAGGATGTCACCTGCCCAACTGCC
GAGGAGACCCTGCCCATTGAACTGGCGTTGGAAGCACGGCAGCAGAATAA
ATATGAGAACTACAGCACCAGCTTCTTCATCAGGGACATCATCAAACCAG
ACCCGCCCAAGAACTTGCAGATGAAGCCTTTGAAGAACTCACAGGTGGAG
GTCAGCTGGGAGTACCCTGACTCCTGGAGCACTCCCCATTCCTACTTCTC
CCTCAAGTTCTTTGTTCGAATCCAGCGCAAGAAAGAAAAGATGAAGGAGA
CAGAGGAGGGGTGTAACCAGAAAGGTGCGTTCCTCGTAGAGAAGACATCT
ACCGAAGTCCAATGCAAAGGCGGGAATGTCTGCGTGCAAGCTCAGGATCG
CTATTACAATTCCTCATGCAGCAAGTGGGCATGTGTTCCCTGCAGAGTCC
GATCGGTTCCTGGAGTAGGGGTACCTGGAGTGGGCAGGGTCATACCGGTC
TCTGGACCTGCCAGGTGTCTTAGCCAGTCCCGAAACCTGCTGAAGACCAC
AGATGACATGGTGAAGACGGCCAGAGAAAAGCTGAAACATTATTCCTGCA
CTGCTGAAGACATCGATCATGAAGACATCACACGGGACCAAACCAGCACA
TTGAAGACCTGTTTACCACTGGAACTACACAAGAACGAGAGTTGCCTGGC
TACTAGAGAGACTTCTTCCACAACAAGAGGGAGCTGCCTGCCCCCACAGA
AGACGTCTTTGATGATGACCCTGTGCCTTGGTAGCATCTATGAGGACTTG
AAGATGTACCAGACAGAGTTCCAGGCCATCAACGCAGCACTTCAGAATCA
CAACCATCAGCAGATCATTCTAGACAAGGGCATGCTGGTGGCCATCGATG
AGCTGATGCAGTCTCTGAATCATAATGGCGAGACTCTGCGCCAGAAACCT
CCTGTGGGAGAAGCAGACCCTTACAGAGTGAAAATGAAGCTCTGCATCCT
GCTTCACGCCTTCAGCACCCGCGTCGTGACCATCAACAGGGTGATGGGCT
ATCTGAGCTCCGCCGGAGGTGGAGGGTCCATAAGTAGTGGACTGCTTTCT
GGCCGAAGTGATAATCACGGCGGCGGCGGTTCCAACACACTTGTGCTCTT
TGGGGCAGGATTCGGCGCAGTAATAACAGTCGTCGTCATCGTTGTCATCA
TCAAATGCTTCTGTAAGCACAGAAGCTGTTTCAGAAGAAGAAATGAGGCA
AGCAGAGAAACAAACAACAGCCTTACCTTCGGGCCTGAAGAAGCATTAGC
TGAACAGACCGTCTTCCTTTAG
IL-12p70-L3-TM
(SEQ ID NO: 21)
ATGTGTCCTCAGAAGCTAACCATCTCCTGGTTTGCCATCGTTTTGCTGGT
GTCTCCACTCATGGCCATGTGGGAGCTGGAGAAAGACGTTTATGTTGTAG
AGGTGGACTGGACTCCCGATGCCCCTGGAGAAACAGTGAACCTCACCTGT
GACACGCCTGAAGAAGATGACATCACCTGGACCTCAGACCAGAGACATGG
AGTCATAGGCTCTGGAAAGACCCTGACCATCACTGTCAAAGAGTTTCTAG
ATGCTGGCCAGTACACCTGCCACAAAGGAGGCGAGACTCTGAGCCACTCA
CATCTGCTGCTCCACAAGAAGGAAAATGGAATTTGGTCCACTGAAATTTT
AAAAAATTTCAAAAACAAGACTTTCCTGAAGTGTGAAGCACCAAATTACT
CCGGACGGTTCACGTGCTCATGGCTGGTGCAAAGAAACATGGACTTGAAG
TTCAACATCAAGAGCAGTAGCAGTTCCCCTGACTCTCGGGCAGTGACATG
TGGAATGGCGTCTCTGTCTGCAGAGAAGGTCACACTGGACCAAAGGGACT
ATGAGAAGTATTCAGTGTCCTGCCAGGAGGATGTCACCTGCCCAACTGCC
GAGGAGACCCTGCCCATTGAACTGGCGTTGGAAGCACGGCAGCAGAATAA
ATATGAGAACTACAGCACCAGCTTCTTCATCAGGGACATCATCAAACCAG
ACCCGCCCAAGAACTTGCAGATGAAGCCTTTGAAGAACTCACAGGTGGAG
GTCAGCTGGGAGTACCCTGACTCCTGGAGCACTCCCCATTCCTACTTCTC
CCTCAAGTTCTTTGTTCGAATCCAGCGCAAGAAAGAAAAGATGAAGGAGA
CAGAGGAGGGGTGTAACCAGAAAGGTGCGTTCCTCGTAGAGAAGACATCT
ACCGAAGTCCAATGCAAAGGCGGGAATGTCTGCGTGCAAGCTCAGGATCG
CTATTACAATTCCTCATGCAGCAAGTGGGCATGTGTTCCCTGCAGAGTCC
GATCGGTTCCTGGAGTAGGGGTACCTGGAGTGGGCAGGGTCATACCGGTC
TCTGGACCTGCCAGGTGTCTTAGCCAGTCCCGAAACCTGCTGAAGACCAC
AGATGACATGGTGAAGACGGCCAGAGAAAAGCTGAAACATTATTCCTGCA
CTGCTGAAGACATCGATCATGAAGACATCACACGGGACCAAACCAGCACA
TTGAAGACCTGTTTACCACTGGAACTACACAAGAACGAGAGTTGCCTGGC
TACTAGAGAGACTTCTTCCACAACAAGAGGGAGCTGCCTGCCCCCACAGA
AGACGTCTTTGATGATGACCCTGTGCCTTGGTAGCATCTATGAGGACTTG
AAGATGTACCAGACAGAGTTCCAGGCCATCAACGCAGCACTTCAGAATCA
CAACCATCAGCAGATCATTCTAGACAAGGGCATGCTGGTGGCCATCGATG
AGCTGATGCAGTCTCTGAATCATAATGGCGAGACTCTGCGCCAGAAACCT
CCTGTGGGAGAAGCAGACCCTTACAGAGTGAAAATGAAGCTCTGCATCCT
GCTTCACGCCTTCAGCACCCGCGTCGTGACCATCAACAGGGTGATGGGCT
ATCTGAGCTCCGCCGGCGGCGGCGGGTCCGTTCCATTGTCCCTTTATTCC
GGGGGTGGTATATCATCAGGACTGCTCAGCGGACGATCTGACAATCACGG
TGGAGGCGGAAGTAACACACTTGTGCTCTTTGGGGCAGGATTCGGCGCAG
TAATAACAGTCGTCGTCATCGTTGTCATCATCAAATGCTTCTGTAAGCAC
AGAAGCTGTTTCAGAAGAAGAAATGAGGCAAGCAGAGAAACAAACAACAG
CCTTACCTTCGGGCCTGAAGAAGCATTAGCTGAACAGACCGTCTTCCTTT
AG
IL-12p70-L4-TM
(SEQ ID NO: 22)
ATGTGTCCTCAGAAGCTAACCATCTCCTGGTTTGCCATCGTTTTGCTGGT
GTCTCCACTCATGGCCATGTGGGAGCTGGAGAAAGACGTTTATGTTGTAG
AGGTGGACTGGACTCCCGATGCCCCTGGAGAAACAGTGAACCTCACCTGT
GACACGCCTGAAGAAGATGACATCACCTGGACCTCAGACCAGAGACATGG
AGTCATAGGCTCTGGAAAGACCCTGACCATCACTGTCAAAGAGTTTCTAG
ATGCTGGCCAGTACACCTGCCACAAAGGAGGCGAGACTCTGAGCCACTCA
CATCTGCTGCTCCACAAGAAGGAAAATGGAATTTGGTCCACTGAAATTTT
AAAAAATTTCAAAAACAAGACTTTCCTGAAGTGTGAAGCACCAAATTACT
CCGGACGGTTCACGTGCTCATGGCTGGTGCAAAGAAACATGGACTTGAAG
TTCAACATCAAGAGCAGTAGCAGTTCCCCTGACTCTCGGGCAGTGACATG
TGGAATGGCGTCTCTGTCTGCAGAGAAGGTCACACTGGACCAAAGGGACT
ATGAGAAGTATTCAGTGTCCTGCCAGGAGGATGTCACCTGCCCAACTGCC
GAGGAGACCCTGCCCATTGAACTGGCGTTGGAAGCACGGCAGCAGAATAA
ATATGAGAACTACAGCACCAGCTTCTTCATCAGGGACATCATCAAACCAG
ACCCGCCCAAGAACTTGCAGATGAAGCCTTTGAAGAACTCACAGGTGGAG
GTCAGCTGGGAGTACCCTGACTCCTGGAGCACTCCCCATTCCTACTTCTC
CCTCAAGTTCTTTGTTCGAATCCAGCGCAAGAAAGAAAAGATGAAGGAGA
CAGAGGAGGGGTGTAACCAGAAAGGTGCGTTCCTCGTAGAGAAGACATCT
ACCGAAGTCCAATGCAAAGGCGGGAATGTCTGCGTGCAAGCTCAGGATCG
CTATTACAATTCCTCATGCAGCAAGTGGGCATGTGTTCCCTGCAGAGTCC
GATCGGTTCCTGGAGTAGGGGTACCTGGAGTGGGCAGGGTCATACCGGTC
TCTGGACCTGCCAGGTGTCTTAGCCAGTCCCGAAACCTGCTGAAGACCAC
AGATGACATGGTGAAGACGGCCAGAGAAAAGCTGAAACATTATTCCTGCA
CTGCTGAAGACATCGATCATGAAGACATCACACGGGACCAAACCAGCACA
TTGAAGACCTGTTTACCACTGGAACTACACAAGAACGAGAGTTGCCTGGC
TACTAGAGAGACTTCTTCCACAACAAGAGGGAGCTGCCTGCCCCCACAGA
AGACGTCTTTGATGATGACCCTGTGCCTTGGTAGCATCTATGAGGACTTG
AAGATGTACCAGACAGAGTTCCAGGCCATCAACGCAGCACTTCAGAATCA
CAACCATCAGCAGATCATTCTAGACAAGGGCATGCTGGTGGCCATCGATG
AGCTGATGCAGTCTCTGAATCATAATGGCGAGACTCTGCGCCAGAAACCT
CCTGTGGGAGAAGCAGACCCTTACAGAGTGAAAATGAAGCTCTGCATCCT
GCTTCACGCCTTCAGCACCCGCGTCGTGACCATCAACAGGGTGATGGGCT
ATCTGAGCTCCGCCGGCGGCGGCGGGTCCCATCCAGTTGGACTGCTGGCC
CGCGGTGGGGGCCACCCGGTGGGGCTGTTGGCAAGAGGGGGCTCAGGGCG
CAGCGCTGGTGGTAGTGGCAGAAGTGCGGGTGGAGGCGGAAGTAACACAC
TTGTGCTCTTTGGGGCAGGATTCGGCGCAGTAATAACAGTCGTCGTCATC
GTTGTCATCATCAAATGCTTCTGTAAGCACAGAAGCTGTTTCAGAAGAAG
AAATGAGGCAAGCAGAGAAACAAACAACAGCCTTACCTTCGGGCCTGAAG
AAGCATTAGCTGAACAGACCGTCTTCCTTTAG
IL-12p70-L4s-TM
(SEQ ID NO: 23)
ATGTGTCCTCAGAAGCTAACCATCTCCTGGTTTGCCATCGTTTTGCTGGT
GTCTCCACTCATGGCCATGTGGGAGCTGGAGAAAGACGTTTATGTTGTAG
AGGTGGACTGGACTCCCGATGCCCCTGGAGAAACAGTGAACCTCACCTGT
GACACGCCTGAAGAAGATGACATCACCTGGACCTCAGACCAGAGACATGG
AGTCATAGGCTCTGGAAAGACCCTGACCATCACTGTCAAAGAGTTTCTAG
ATGCTGGCCAGTACACCTGCCACAAAGGAGGCGAGACTCTGAGCCACTCA
CATCTGCTGCTCCACAAGAAGGAAAATGGAATTTGGTCCACTGAAATTTT
AAAAAATTTCAAAAACAAGACTTTCCTGAAGTGTGAAGCACCAAATTACT
CCGGACGGTTCACGTGCTCATGGCTGGTGCAAAGAAACATGGACTTGAAG
TTCAACATCAAGAGCAGTAGCAGTTCCCCTGACTCTCGGGCAGTGACATG
TGGAATGGCGTCTCTGTCTGCAGAGAAGGTCACACTGGACCAAAGGGACT
ATGAGAAGTATTCAGTGTCCTGCCAGGAGGATGTCACCTGCCCAACTGCC
GAGGAGACCCTGCCCATTGAACTGGCGTTGGAAGCACGGCAGCAGAATAA
ATATGAGAACTACAGCACCAGCTTCTTCATCAGGGACATCATCAAACCAG
ACCCGCCCAAGAACTTGCAGATGAAGCCTTTGAAGAACTCACAGGTGGAG
GTCAGCTGGGAGTACCCTGACTCCTGGAGCACTCCCCATTCCTACTTCTC
CCTCAAGTTCTTTGTTCGAATCCAGCGCAAGAAAGAAAAGATGAAGGAGA
CAGAGGAGGGGTGTAACCAGAAAGGTGCGTTCCTCGTAGAGAAGACATCT
ACCGAAGTCCAATGCAAAGGCGGGAATGTCTGCGTGCAAGCTCAGGATCG
CTATTACAATTCCTCATGCAGCAAGTGGGCATGTGTTCCCTGCAGAGTCC
GATCGGTTCCTGGAGTAGGGGTACCTGGAGTGGGCAGGGTCATACCGGTC
TCTGGACCTGCCAGGTGTCTTAGCCAGTCCCGAAACCTGCTGAAGACCAC
AGATGACATGGTGAAGACGGCCAGAGAAAAGCTGAAACATTATTCCTGCA
CTGCTGAAGACATCGATCATGAAGACATCACACGGGACCAAACCAGCACA
TTGAAGACCTGTTTACCACTGGAACTACACAAGAACGAGAGTTGCCTGGC
TACTAGAGAGACTTCTTCCACAACAAGAGGGAGCTGCCTGCCCCCACAGA
AGACGTCTTTGATGATGACCCTGTGCCTTGGTAGCATCTATGAGGACTTG
AAGATGTACCAGACAGAGTTCCAGGCCATCAACGCAGCACTTCAGAATCA
CAACCATCAGCAGATCATTCTAGACAAGGGCATGCTGGTGGCCATCGATG
AGCTGATGCAGTCTCTGAATCATAATGGCGAGACTCTGCGCCAGAAACCT
CCTGTGGGAGAAGCAGACCCTTACAGAGTGAAAATGAAGCTCTGCATCCT
GCTTCACGCCTTCAGCACCCGCGTCGTGACCATCAACAGGGTGATGGGCT
ATCTGAGCTCCGCCGGCGGCGGCGGGTCCCATCCAGTTGGACTGCTGGCC
CGCGGTGGAGGCGGAAGTAACACACTTGTGCTCTTTGGGGCAGGATTCGG
CGCAGTAATAACAGTCGTCGTCATCGTTGTCATCATCAAATGCTTCTGTA
AGCACAGAAGCTGTTTCAGAAGAAGAAATGAGGCAAGCAGAGAAACAAAC
AACAGCCTTACCTTCGGGCCTGAAGAAGCATTAGCTGAACAGACCGTCTT
CCTTTAG
IL-12p70-L5-TM
(SEQ ID NO: 24)
ATGTGTCCTCAGAAGCTAACCATCTCCTGGTTTGCCATCGTTTTGCTGGT
GTCTCCACTCATGGCCATGTGGGAGCTGGAGAAAGACGTTTATGTTGTAG
AGGTGGACTGGACTCCCGATGCCCCTGGAGAAACAGTGAACCTCACCTGT
GACACGCCTGAAGAAGATGACATCACCTGGACCTCAGACCAGAGACATGG
AGTCATAGGCTCTGGAAAGACCCTGACCATCACTGTCAAAGAGTTTCTAG
ATGCTGGCCAGTACACCTGCCACAAAGGAGGCGAGACTCTGAGCCACTCA
CATCTGCTGCTCCACAAGAAGGAAAATGGAATTTGGTCCACTGAAATTTT
AAAAAATTTCAAAAACAAGACTTTCCTGAAGTGTGAAGCACCAAATTACT
CCGGACGGTTCACGTGCTCATGGCTGGTGCAAAGAAACATGGACTTGAAG
TTCAACATCAAGAGCAGTAGCAGTTCCCCTGACTCTCGGGCAGTGACATG
TGGAATGGCGTCTCTGTCTGCAGAGAAGGTCACACTGGACCAAAGGGACT
ATGAGAAGTATTCAGTGTCCTGCCAGGAGGATGTCACCTGCCCAACTGCC
GAGGAGACCCTGCCCATTGAACTGGCGTTGGAAGCACGGCAGCAGAATAA
ATATGAGAACTACAGCACCAGCTTCTTCATCAGGGACATCATCAAACCAG
ACCCGCCCAAGAACTTGCAGATGAAGCCTTTGAAGAACTCACAGGTGGAG
GTCAGCTGGGAGTACCCTGACTCCTGGAGCACTCCCCATTCCTACTTCTC
CCTCAAGTTCTTTGTTCGAATCCAGCGCAAGAAAGAAAAGATGAAGGAGA
CAGAGGAGGGGTGTAACCAGAAAGGTGCGTTCCTCGTAGAGAAGACATCT
ACCGAAGTCCAATGCAAAGGCGGGAATGTCTGCGTGCAAGCTCAGGATCG
CTATTACAATTCCTCATGCAGCAAGTGGGCATGTGTTCCCTGCAGAGTCC
GATCGGTTCCTGGAGTAGGGGTACCTGGAGTGGGCAGGGTCATACCGGTC
TCTGGACCTGCCAGGTGTCTTAGCCAGTCCCGAAACCTGCTGAAGACCAC
AGATGACATGGTGAAGACGGCCAGAGAAAAGCTGAAACATTATTCCTGCA
CTGCTGAAGACATCGATCATGAAGACATCACACGGGACCAAACCAGCACA
TTGAAGACCTGTTTACCACTGGAACTACACAAGAACGAGAGTTGCCTGGC
TACTAGAGAGACTTCTTCCACAACAAGAGGGAGCTGCCTGCCCCCACAGA
AGACGTCTTTGATGATGACCCTGTGCCTTGGTAGCATCTATGAGGACTTG
AAGATGTACCAGACAGAGTTCCAGGCCATCAACGCAGCACTTCAGAATCA
CAACCATCAGCAGATCATTCTAGACAAGGGCATGCTGGTGGCCATCGATG
AGCTGATGCAGTCTCTGAATCATAATGGCGAGACTCTGCGCCAGAAACCT
CCTGTGGGAGAAGCAGACCCTTACAGAGTGAAAATGAAGCTCTGCATCCT
GCTTCACGCCTTCAGCACCCGCGTCGTGACCATCAACAGGGTGATGGGCT
ATCTGAGCTCCGCCGGCGGCGGCGGGTCCCTTGCTCAGGCGGTGCGATCC
AGCGGTGGAGGCGGAAGTAACACACTTGTGCTCTTTGGGGCAGGATTCGG
CGCAGTAATAACAGTCGTCGTCATCGTTGTCATCATCAAATGCTTCTGTA
AGCACAGAAGCTGTTTCAGAAGAAGAAATGAGGCAAGCAGAGAAACAAAC
AACAGCCTTACCTTCGGGCCTGAAGAAGCATTAGCTGAACAGACCGTCTT
CCTTTAG

SEQUENCE LISTING

| SEQ ID NO | |
|---|---|
| or | |
| SEQ NO | Sequence |
| SEQ ID | GGGGSGGGGSGGGGS |
| NO: 1 | |
| SEQ ID | GGGGSGGGGSGGGGSGGGS |
| NO: 2 | |
| SEQ ID | GGGGSISSGLLSGRSDNHGGGGS |
| NO: 3 | |
| SEQ ID | GGGGSVPLSLYSGGGISSGLLSGRSDNHGGGGS |
| NO: 4 | |
| SEQ ID | GGGGSHPVGLLARGGGHPVGLLARGGSGRSAGGSGRSAGGGGS |
| NO: 5 | |
| SEQ ID | GGGGSHPVGLLARGGGGS |
| NO: 6 | |
| SEQ ID | GGGGSLAQAVRSSGGGGS |
| NO: 7 | |
| SEQ ID | ISSGLLSGRSDNH |
| NO: 8 | |
| SEQ ID | VPLSLYSGGGISSGLLSGRSDNH |
| NO: 9 | |
| SEQ ID | HPVGLLAR |
| NO: 10 | |
| SEQ ID | SGRSA |
| NO: 11 | |
| SEQ ID | LAQAVRSS |
| NO: 12 | |
| SEQ ID | LSGRSDNH |
| NO: 13 | |
| SEQ ID | ISSGLLSS |
| NO: 14 | |
| SEQ NO: | SGR |
| 15 | |
| SEQ ID | VPLSLYSG |
| NO: 16 | |
| SEQ ID | ATGTGTCCTCAGAAGCTAACCATCTCCTGGTTTGCCATCGTTTTGCT |
| NO: 17 | GGTGTCTCCACTCATGGCCATGTGGGAGCTGGAGAAAGACGTTTAT |
| | GTTGTAGAGGTGGACTGGACTCCCGATGCCCCTGGAGAAACAGTGA |
| | ACCTCACCTGTGACACGCCTGAAGAAGATGACATCACCTGGACCTC |
| | AGACCAGAGACATGGAGTCATAGGCTCTGGAAAGACCCTGACCATC |
| | ACTGTCAAAGAGTTTCTAGATGCTGGCCAGTACACCTGCCACAAAG |
| | GAGGCGAGACTCTGAGCCACTCACATCTGCTGCTCCACAAGAAGGA |
| | AAATGGAATTTGGTCCACTGAAATTTTAAAAAATTTCAAAAACAAG |
| | ACTTTCCTGAAGTGTGAAGCACCAAATTACTCCGGACGGTTCACGT |
| | GCTCATGGCTGGTGCAAAGAAACATGGACTTGAAGTTCAACATCAA |
| | GAGCAGTAGCAGTTCCCCTGACTCTCGGGCAGTGACATGTGGAATG |
| | GCGTCTCTGTCTGCAGAGAAGGTCACACTGGACCAAAGGGACTATG |
| | AGAAGTATTCAGTGTCCTGCCAGGAGGATGTCACCTGCCCAACTGC |
| | CGAGGAGACCCTGCCCATTGAACTGGCGTTGGAAGCACGGCAGCAG |
| | AATAAATATGAGAACTACAGCACCAGCTTCTTCATCAGGGACATCA |
| | TCAAACCAGACCCGCCCAAGAACTTGCAGATGAAGCCTTTGAAGAA |
| | CTCACAGGTGGAGGTCAGCTGGGAGTACCCTGACTCCTGGAGCACT |
| | CCCCATTCCTACTTCTCCCTCAAGTTCTTTGTTCGAATCCAGCGCAA |
| | GAAAGAAAAGATGAAGGAGACAGAGGAGGGGTGTAACCAGAAAG |
| | GTGCGTTCCTCGTAGAGAAGACATCTACCGAAGTCCAATGCAAAGG |
| | CGGGAATGTCTGCGTGCAAGCTCAGGATCGCTATTACAATTCCTCAT |
| | GCAGCAAGTGGGCATGTGTTCCCTGCAGAGTCCGATCGGTTCCTGG |
| | AGTAGGGGTACCTGGAGTGGGCAGGGTCATACCGGTCTCTGGACCT |
| | GCCAGGTGTCTTAGCCAGTCCCGAAACCTGCTGAAGACCACAGATG |
| | ACATGGTGAAGACGGCCAGAGAAAAGCTGAAACATTATTCCTGCAC |
| | TGCTGAAGACATCGATCATGAAGACATCACACGGGACCAAACCAGC |
| | ACATTGAAGACCTGTTTACCACTGGAACTACACAAGAACGAGAGTT |
| | GCCTGGCTACTAGAGAGACTTCTTCCACAACAAGAGGGAGCTGCCT |
| | GCCCCCACAGAAGACGTCTTTGATGATGACCCTGTGCCTTGGTAGC |
| | ATCTATGAGGACTTGAAGATGTACCAGACAGAGTTCCAGGCCATCA |
| | ACGCAGCACTTCAGAATCACAACCATCAGCAGATCATTCTAGACAA |
| | GGGCATGCTGGTGGCCATCGATGAGCTGATGCAGTCTCTGAATCAT |
| | AATGGCGAGACTCTGCGCCAGAAACCTCCTGTGGGAGAAGCAGACC |
| | CTTACAGAGTGAAAATGAAGCTCTGCATCCTGCTTCACGCCTTCAGC |
| | ACCCGCGTCGTGACCATCAACAGGGTGATGGGCTATCTGAGCTCCG |
| | CCTGA |
| SEQ ID | ATGTGTCCTCAGAAGCTAACCATCTCCTGGTTTGCCATCGTTTTGCT |
| NO: 18 | GGTGTCTCCACTCATGGCCATGTGGGAGCTGGAGAAAGACGTTTAT |
| | GTTGTAGAGGTGGACTGGACTCCCGATGCCCCTGGAGAAACAGTGA |
| | ACCTCACCTGTGACACGCCTGAAGAAGATGACATCACCTGGACCTC |
| | AGACCAGAGACATGGAGTCATAGGCTCTGGAAAGACCCTGACCATC |
| | ACTGTCAAAGAGTTTCTAGATGCTGGCCAGTACACCTGCCACAAAG |
| | GAGGCGAGACTCTGAGCCACTCACATCTGCTGCTCCACAAGAAGGA |
| | AAATGGAATTTGGTCCACTGAAATTTTAAAAAATTTCAAAAACAAG |
| | ACTTTCCTGAAGTGTGAAGCACCAAATTACTCCGGACGGTTCACGT |
| | GCTCATGGCTGGTGCAAAGAAACATGGACTTGAAGTTCAACATCAA |
| | GAGCAGTAGCAGTTCCCCTGACTCTCGGGCAGTGACATGTGGAATG |
| | GCGTCTCTGTCTGCAGAGAAGGTCACACTGGACCAAAGGGACTATG |
| | AGAAGTATTCAGTGTCCTGCCAGGAGGATGTCACCTGCCCAACTGC |
| | CGAGGAGACCCTGCCCATTGAACTGGCGTTGGAAGCACGGCAGCAG |
| | AATAAATATGAGAACTACAGCACCAGCTTCTTCATCAGGGACATCA |
| | TCAAACCAGACCCGCCCAAGAACTTGCAGATGAAGCCTTTGAAGAA |
| | CTCACAGGTGGAGGTCAGCTGGGAGTACCCTGACTCCTGGAGCACT |
| | CCCCATTCCTACTTCTCCCTCAAGTTCTTTGTTCGAATCCAGCGCAA |
| | GAAAGAAAAGATGAAGGAGACAGAGGAGGGGTGTAACCAGAAAG |
| | GTGCGTTCCTCGTAGAGAAGACATCTACCGAAGTCCAATGCAAAGG |
| | CGGGAATGTCTGCGTGCAAGCTCAGGATCGCTATTACAATTCCTCAT |
| | GCAGCAAGTGGGCATGTGTTCCCTGCAGAGTCCGATCGGTTCCTGG |
| | AGTAGGGGTACCTGGAGTGGGCAGGGTCATACCGGTCTCTGGACCT |
| | GCCAGGTGTCTTAGCCAGTCCCGAAACCTGCTGAAGACCACAGATG |
| | ACATGGTGAAGACGGCCAGAGAAAAGCTGAAACATTATTCCTGCAC |
| | TGCTGAAGACATCGATCATGAAGACATCACACGGGACCAAACCAGC |
| | ACATTGAAGACCTGTTTACCACTGGAACTACACAAGAACGAGAGTT |
| | GCCTGGCTACTAGAGAGACTTCTTCCACAACAAGAGGGAGCTGCCT |
| | GCCCCCACAGAAGACGTCTTTGATGATGACCCTGTGCCTTGGTAGC |
| | ATCTATGAGGACTTGAAGATGTACCAGACAGAGTTCCAGGCCATCA |
| | ACGCAGCACTTCAGAATCACAACCATCAGCAGATCATTCTAGACAA |
| | GGGCATGCTGGTGGCCATCGATGAGCTGATGCAGTCTCTGAATCAT |
| | AATGGCGAGACTCTGCGCCAGAAACCTCCTGTGGGAGAAGCAGACC |
| | CTTACAGAGTGAAAATGAAGCTCTGCATCCTGCTTCACGCCTTCAGC |
| | ACCCGCGTCGTGACCATCAACAGGGTGATGGGCTATCTGAGCTCCG |
| | CCGGGGGGGGAGGCAGTGGAGGCGGTGGTTCAGGTGGTGGTGGGT |
| | CCAACACACTTGTGCTCTTTGGGGCAGGATTCGGCGCAGTAATAAC |
| | AGTCGTCGTCATCGTTGTCATCATCAAATGCTTCTGTAAGCACAGAA |
| | GCTGTTTCAGAAGAAGAAATGAGGCAAGCAGAGAAACAAACAACA |
| | GCCTTACCTTCGGGCCTGAAGAAGCATTAGCTGAACAGACCGTCTT |
| | CCTTTAG |
| SEQ ID | ATGTGTCCTCAGAAGCTAACCATCTCCTGGTTTGCCATCGTTTTGCT |
| NO: 19 | GGTGTCTCCACTCATGGCCATGTGGGAGCTGGAGAAAGACGTTTAT |
| | GTTGTAGAGGTGGACTGGACTCCCGATGCCCCTGGAGAAACAGTGA |
| | ACCTCACCTGTGACACGCCTGAAGAAGATGACATCACCTGGACCTC |
| | AGACCAGAGACATGGAGTCATAGGCTCTGGAAAGACCCTGACCATC |
| | ACTGTCAAAGAGTTTCTAGATGCTGGCCAGTACACCTGCCACAAAG |
| | GAGGCGAGACTCTGAGCCACTCACATCTGCTGCTCCACAAGAAGGA |
| | AAATGGAATTTGGTCCACTGAAATTTTAAAAAATTTCAAAAACAAG |
| | ACTTTCCTGAAGTGTGAAGCACCAAATTACTCCGGACGGTTCACGT |
| | GCTCATGGCTGGTGCAAAGAAACATGGACTTGAAGTTCAACATCAA |
| | GAGCAGTAGCAGTTCCCCTGACTCTCGGGCAGTGACATGTGGAATG |
| | GCGTCTCTGTCTGCAGAGAAGGTCACACTGGACCAAAGGGACTATG |
| | AGAAGTATTCAGTGTCCTGCCAGGAGGATGTCACCTGCCCAACTGC |
| | CGAGGAGACCCTGCCCATTGAACTGGCGTTGGAAGCACGGCAGCAG |
| | AATAAATATGAGAACTACAGCACCAGCTTCTTCATCAGGGACATCA |
| | TCAAACCAGACCCGCCCAAGAACTTGCAGATGAAGCCTTTGAAGAA |
| | CTCACAGGTGGAGGTCAGCTGGGAGTACCCTGACTCCTGGAGCACT |
| | CCCCATTCCTACTTCTCCCTCAAGTTCTTTGTTCGAATCCAGCGCAA |
| | GAAAGAAAAGATGAAGGAGACAGAGGAGGGGTGTAACCAGAAAG |
| | GTGCGTTCCTCGTAGAGAAGACATCTACCGAAGTCCAATGCAAAGG |
| | CGGGAATGTCTGCGTGCAAGCTCAGGATCGCTATTACAATTCCTCAT |
| | GCAGCAAGTGGGCATGTGTTCCCTGCAGAGTCCGATCGGTTCCTGG |
| | AGTAGGGGTACCTGGAGTGGGCAGGGTCATACCGGTCTCTGGACCT |
| | GCCAGGTGTCTTAGCCAGTCCCGAAACCTGCTGAAGACCACAGATG |
| | ACATGGTGAAGACGGCCAGAGAAAAGCTGAAACATTATTCCTGCAC |
| | TGCTGAAGACATCGATCATGAAGACATCACACGGGACCAAACCAGC |
| | ACATTGAAGACCTGTTTACCACTGGAACTACACAAGAACGAGAGTT |
| | GCCTGGCTACTAGAGAGACTTCTTCCACAACAAGAGGGAGCTGCCT |
| | GCCCCCACAGAAGACGTCTTTGATGATGACCCTGTGCCTTGGTAGC |
| | ATCTATGAGGACTTGAAGATGTACCAGACAGAGTTCCAGGCCATCA |
| | ACGCAGCACTTCAGAATCACAACCATCAGCAGATCATTCTAGACAA |
| | GGGCATGCTGGTGGCCATCGATGAGCTGATGCAGTCTCTGAATCAT |
| | AATGGCGAGACTCTGCGCCAGAAACCTCCTGTGGGAGAAGCAGACC |
| | CTTACAGAGTGAAAATGAAGCTCTGCATCCTGCTTCACGCCTTCAGC |
| | ACCCGCGTCGTGACCATCAACAGGGTGATGGGCTATCTGAGCTCCG |
| | CCGGGGGGGGAGGCAGTGGAGGCGGTGGTTCAGGTGGTGGTGGGT |
| | CCAACACACTTGTGCTCTTTGGGGCAGGATTCGGCGCAGTAATAAC |
| | AGTCGTCGTCATCGTTGTCATCATCAAATGCTTCTGTAAGCACAGAA |
| | GCTGTTTCAGAAGAAGAAATGAGGCAAGCAGAGAAACAAACAACA |
| | GCCTTACCTTCGGGCCTGAAGAAGCATTAGCTGAACAGACCGTCTT |
| | CCTTTAG |
| SEQ ID | ATGTGTCCTCAGAAGCTAACCATCTCCTGGTTTGCCATCGTTTTGCT |
| NO: 20 | GGTGTCTCCACTCATGGCCATGTGGGAGCTGGAGAAAGACGTTTAT |
| | GTTGTAGAGGTGGACTGGACTCCCGATGCCCCTGGAGAAACAGTGA |
| | ACCTCACCTGTGACACGCCTGAAGAAGATGACATCACCTGGACCTC |
| | AGACCAGAGACATGGAGTCATAGGCTCTGGAAAGACCCTGACCATC |
| | ACTGTCAAAGAGTTTCTAGATGCTGGCCAGTACACCTGCCACAAAG |
| | GAGGCGAGACTCTGAGCCACTCACATCTGCTGCTCCACAAGAAGGA |
| | AAATGGAATTTGGTCCACTGAAATTTTAAAAAATTTCAAAAACAAG |
| | ACTTTCCTGAAGTGTGAAGCACCAAATTACTCCGGACGGTTCACGT |
| | GCTCATGGCTGGTGCAAAGAAACATGGACTTGAAGTTCAACATCAA |
| | GAGCAGTAGCAGTTCCCCTGACTCTCGGGCAGTGACATGTGGAATG |
| | GCGTCTCTGTCTGCAGAGAAGGTCACACTGGACCAAAGGGACTATG |
| | AGAAGTATTCAGTGTCCTGCCAGGAGGATGTCACCTGCCCAACTGC |
| | CGAGGAGACCCTGCCCATTGAACTGGCGTTGGAAGCACGGCAGCAG |
| | AATAAATATGAGAACTACAGCACCAGCTTCTTCATCAGGGACATCA |
| | TCAAACCAGACCCGCCCAAGAACTTGCAGATGAAGCCTTTGAAGAA |
| | CTCACAGGTGGAGGTCAGCTGGGAGTACCCTGACTCCTGGAGCACT |
| | CCCCATTCCTACTTCTCCCTCAAGTTCTTTGTTCGAATCCAGCGCAA |
| | GAAAGAAAAGATGAAGGAGACAGAGGAGGGGTGTAACCAGAAAG |
| | GTGCGTTCCTCGTAGAGAAGACATCTACCGAAGTCCAATGCAAAGG |
| | CGGGAATGTCTGCGTGCAAGCTCAGGATCGCTATTACAATTCCTCAT |
| | GCAGCAAGTGGGCATGTGTTCCCTGCAGAGTCCGATCGGTTCCTGG |
| | AGTAGGGGTACCTGGAGTGGGCAGGGTCATACCGGTCTCTGGACCT |
| | GCCAGGTGTCTTAGCCAGTCCCGAAACCTGCTGAAGACCACAGATG |
| | ACATGGTGAAGACGGCCAGAGAAAAGCTGAAACATTATTCCTGCAC |
| | TGCTGAAGACATCGATCATGAAGACATCACACGGGACCAAACCAGC |
| | ACATTGAAGACCTGTTTACCACTGGAACTACACAAGAACGAGAGTT |
| | GCCTGGCTACTAGAGAGACTTCTTCCACAACAAGAGGGAGCTGCCT |
| | GCCCCCACAGAAGACGTCTTTGATGATGACCCTGTGCCTTGGTAGC |
| | ATCTATGAGGACTTGAAGATGTACCAGACAGAGTTCCAGGCCATCA |
| | ACGCAGCACTTCAGAATCACAACCATCAGCAGATCATTCTAGACAA |
| | GGGCATGCTGGTGGCCATCGATGAGCTGATGCAGTCTCTGAATCAT |
| | AATGGCGAGACTCTGCGCCAGAAACCTCCTGTGGGAGAAGCAGACC |
| | CTTACAGAGTGAAAATGAAGCTCTGCATCCTGCTTCACGCCTTCAGC |
| | ACCCGCGTCGTGACCATCAACAGGGTGATGGGCTATCTGAGCTCCG |
| | CCGGAGGTGGAGGGTCCATAAGTAGTGGACTGCTTTCTGGCCGAAG |
| | TGATAATCACGGCGGCGGCGGTTCCAACACACTTGTGCTCTTTGGG |
| | GCAGGATTCGGCGCAGTAATAACAGTCGTCGTCATCGTTGTCATCAT |
| | CAAATGCTTCTGTAAGCACAGAAGCTGTTTCAGAAGAAGAAATGAG |
| | GCAAGCAGAGAAACAAACAACAGCCTTACCTTCGGGCCTGAAGAA |
| | GCATTAGCTGAACAGACCGTCTTCCTTTAG |
| SEQ ID | ATGTGTCCTCAGAAGCTAACCATCTCCTGGTTTGCCATCGTTTTGCT |
| NO: 21 | GGTGTCTCCACTCATGGCCATGTGGGAGCTGGAGAAAGACGTTTAT |
| | GTTGTAGAGGTGGACTGGACTCCCGATGCCCCTGGAGAAACAGTGA |
| | ACCTCACCTGTGACACGCCTGAAGAAGATGACATCACCTGGACCTC |
| | AGACCAGAGACATGGAGTCATAGGCTCTGGAAAGACCCTGACCATC |
| | ACTGTCAAAGAGTTTCTAGATGCTGGCCAGTACACCTGCCACAAAG |
| | GAGGCGAGACTCTGAGCCACTCACATCTGCTGCTCCACAAGAAGGA |
| | AAATGGAATTTGGTCCACTGAAATTTTAAAAAATTTCAAAAACAAG |
| | ACTTTCCTGAAGTGTGAAGCACCAAATTACTCCGGACGGTTCACGT |
| | GCTCATGGCTGGTGCAAAGAAACATGGACTTGAAGTTCAACATCAA |
| | GAGCAGTAGCAGTTCCCCTGACTCTCGGGCAGTGACATGTGGAATG |
| | GCGTCTCTGTCTGCAGAGAAGGTCACACTGGACCAAAGGGACTATG |
| | AGAAGTATTCAGTGTCCTGCCAGGAGGATGTCACCTGCCCAACTGC |
| | CGAGGAGACCCTGCCCATTGAACTGGCGTTGGAAGCACGGCAGCAG |
| | AATAAATATGAGAACTACAGCACCAGCTTCTTCATCAGGGACATCA |
| | TCAAACCAGACCCGCCCAAGAACTTGCAGATGAAGCCTTTGAAGAA |
| | CTCACAGGTGGAGGTCAGCTGGGAGTACCCTGACTCCTGGAGCACT |
| | CCCCATTCCTACTTCTCCCTCAAGTTCTTTGTTCGAATCCAGCGCAA |
| | GAAAGAAAAGATGAAGGAGACAGAGGAGGGGTGTAACCAGAAAG |
| | GTGCGTTCCTCGTAGAGAAGACATCTACCGAAGTCCAATGCAAAGG |
| | CGGGAATGTCTGCGTGCAAGCTCAGGATCGCTATTACAATTCCTCAT |
| | GCAGCAAGTGGGCATGTGTTCCCTGCAGAGTCCGATCGGTTCCTGG |
| | AGTAGGGGTACCTGGAGTGGGCAGGGTCATACCGGTCTCTGGACCT |
| | GCCAGGTGTCTTAGCCAGTCCCGAAACCTGCTGAAGACCACAGATG |
| | ACATGGTGAAGACGGCCAGAGAAAAGCTGAAACATTATTCCTGCAC |
| | TGCTGAAGACATCGATCATGAAGACATCACACGGGACCAAACCAGC |
| | ACATTGAAGACCTGTTTACCACTGGAACTACACAAGAACGAGAGTT |
| | GCCTGGCTACTAGAGAGACTTCTTCCACAACAAGAGGGAGCTGCCT |
| | GCCCCCACAGAAGACGTCTTTGATGATGACCCTGTGCCTTGGTAGC |
| | ATCTATGAGGACTTGAAGATGTACCAGACAGAGTTCCAGGCCATCA |
| | ACGCAGCACTTCAGAATCACAACCATCAGCAGATCATTCTAGACAA |
| | GGGCATGCTGGTGGCCATCGATGAGCTGATGCAGTCTCTGAATCAT |
| | AATGGCGAGACTCTGCGCCAGAAACCTCCTGTGGGAGAAGCAGACC |
| | CTTACAGAGTGAAAATGAAGCTCTGCATCCTGCTTCACGCCTTCAGC |
| | ACCCGCGTCGTGACCATCAACAGGGTGATGGGCTATCTGAGCTCCG |
| | CCGGCGGCGGCGGGTCCGTTCCATTGTCCCTTTATTCCGGGGGTGGT |
| | ATATCATCAGGACTGCTCAGCGGACGATCTGACAATCACGGTGGAG |
| | GCGGAAGTAACACACTTGTGCTCTTTGGGGCAGGATTCGGCGCAGT |
| | AATAACAGTCGTCGTCATCGTTGTCATCATCAAATGCTTCTGTAAGC |
| | ACAGAAGCTGTTTCAGAAGAAGAAATGAGGCAAGCAGAGAAACAA |
| | ACAACAGCCTTACCTTCGGGCCTGAAGAAGCATTAGCTGAACAGAC |
| | CGTCTTCCTTTAG |
| SEQ ID | ATGTGTCCTCAGAAGCTAACCATCTCCTGGTTTGCCATCGTTTTGCT |
| NO: 22 | GGTGTCTCCACTCATGGCCATGTGGGAGCTGGAGAAAGACGTTTAT |
| | GTTGTAGAGGTGGACTGGACTCCCGATGCCCCTGGAGAAACAGTGA |
| | ACCTCACCTGTGACACGCCTGAAGAAGATGACATCACCTGGACCTC |
| | AGACCAGAGACATGGAGTCATAGGCTCTGGAAAGACCCTGACCATC |
| | ACTGTCAAAGAGTTTCTAGATGCTGGCCAGTACACCTGCCACAAAG |
| | GAGGCGAGACTCTGAGCCACTCACATCTGCTGCTCCACAAGAAGGA |
| | AAATGGAATTTGGTCCACTGAAATTTTAAAAAATTTCAAAAACAAG |
| | ACTTTCCTGAAGTGTGAAGCACCAAATTACTCCGGACGGTTCACGT |
| | GCTCATGGCTGGTGCAAAGAAACATGGACTTGAAGTTCAACATCAA |
| | GAGCAGTAGCAGTTCCCCTGACTCTCGGGCAGTGACATGTGGAATG |
| | GCGTCTCTGTCTGCAGAGAAGGTCACACTGGACCAAAGGGACTATG |
| | AGAAGTATTCAGTGTCCTGCCAGGAGGATGTCACCTGCCCAACTGC |
| | CGAGGAGACCCTGCCCATTGAACTGGCGTTGGAAGCACGGCAGCAG |
| | AATAAATATGAGAACTACAGCACCAGCTTCTTCATCAGGGACATCA |
| | TCAAACCAGACCCGCCCAAGAACTTGCAGATGAAGCCTTTGAAGAA |
| | CTCACAGGTGGAGGTCAGCTGGGAGTACCCTGACTCCTGGAGCACT |
| | CCCCATTCCTACTTCTCCCTCAAGTTCTTTGTTCGAATCCAGCGCAA |
| | GAAAGAAAAGATGAAGGAGACAGAGGAGGGGTGTAACCAGAAAG |
| | GTGCGTTCCTCGTAGAGAAGACATCTACCGAAGTCCAATGCAAAGG |
| | CGGGAATGTCTGCGTGCAAGCTCAGGATCGCTATTACAATTCCTCAT |
| | GCAGCAAGTGGGCATGTGTTCCCTGCAGAGTCCGATCGGTTCCTGG |
| | AGTAGGGGTACCTGGAGTGGGCAGGGTCATACCGGTCTCTGGACCT |
| | GCCAGGTGTCTTAGCCAGTCCCGAAACCTGCTGAAGACCACAGATG |
| | ACATGGTGAAGACGGCCAGAGAAAAGCTGAAACATTATTCCTGCAC |
| | TGCTGAAGACATCGATCATGAAGACATCACACGGGACCAAACCAGC |
| | ACATTGAAGACCTGTTTACCACTGGAACTACACAAGAACGAGAGTT |
| | GCCTGGCTACTAGAGAGACTTCTTCCACAACAAGAGGGAGCTGCCT |
| | GCCCCCACAGAAGACGTCTTTGATGATGACCCTGTGCCTTGGTAGC |
| | ATCTATGAGGACTTGAAGATGTACCAGACAGAGTTCCAGGCCATCA |
| | ACGCAGCACTTCAGAATCACAACCATCAGCAGATCATTCTAGACAA |
| | GGGCATGCTGGTGGCCATCGATGAGCTGATGCAGTCTCTGAATCAT |
| | AATGGCGAGACTCTGCGCCAGAAACCTCCTGTGGGAGAAGCAGACC |
| | CTTACAGAGTGAAAATGAAGCTCTGCATCCTGCTTCACGCCTTCAGC |
| | ACCCGCGTCGTGACCATCAACAGGGTGATGGGCTATCTGAGCTCCG |
| | CCGGCGGCGGCGGGTCCCATCCAGTTGGACTGCTGGCCCGCGGTGG |
| | GGGCCACCCGGTGGGGCTGTTGGCAAGAGGGGGCTCAGGGCGCAG |
| | CGCTGGTGGTAGTGGCAGAAGTGCGGGTGGAGGCGGAAGTAACAC |
| | ACTTGTGCTCTTTGGGGCAGGATTCGGCGCAGTAATAACAGTCGTC |
| | GTCATCGTTGTCATCATCAAATGCTTCTGTAAGCACAGAAGCTGTTT |
| | CAGAAGAAGAAATGAGGCAAGCAGAGAAACAAACAACAGCCTTAC |
| | CTTCGGGCCTGAAGAAGCATTAGCTGAACAGACCGTCTTCCTTTAG |
| SEQ ID | ATGTGTCCTCAGAAGCTAACCATCTCCTGGTTTGCCATCGTTTTGCT |
| NO: 23 | GGTGTCTCCACTCATGGCCATGTGGGAGCTGGAGAAAGACGTTTAT |
| | GTTGTAGAGGTGGACTGGACTCCCGATGCCCCTGGAGAAACAGTGA |
| | ACCTCACCTGTGACACGCCTGAAGAAGATGACATCACCTGGACCTC |
| | AGACCAGAGACATGGAGTCATAGGCTCTGGAAAGACCCTGACCATC |
| | ACTGTCAAAGAGTTTCTAGATGCTGGCCAGTACACCTGCCACAAAG |
| | GAGGCGAGACTCTGAGCCACTCACATCTGCTGCTCCACAAGAAGGA |
| | AAATGGAATTTGGTCCACTGAAATTTTAAAAAATTTCAAAAACAAG |
| | ACTTTCCTGAAGTGTGAAGCACCAAATTACTCCGGACGGTTCACGT |
| | GCTCATGGCTGGTGCAAAGAAACATGGACTTGAAGTTCAACATCAA |
| | GAGCAGTAGCAGTTCCCCTGACTCTCGGGCAGTGACATGTGGAATG |
| | GCGTCTCTGTCTGCAGAGAAGGTCACACTGGACCAAAGGGACTATG |
| | AGAAGTATTCAGTGTCCTGCCAGGAGGATGTCACCTGCCCAACTGC |
| | CGAGGAGACCCTGCCCATTGAACTGGCGTTGGAAGCACGGCAGCAG |
| | AATAAATATGAGAACTACAGCACCAGCTTCTTCATCAGGGACATCA |
| | TCAAACCAGACCCGCCCAAGAACTTGCAGATGAAGCCTTTGAAGAA |
| | CTCACAGGTGGAGGTCAGCTGGGAGTACCCTGACTCCTGGAGCACT |
| | CCCCATTCCTACTTCTCCCTCAAGTTCTTTGTTCGAATCCAGCGCAA |
| | GAAAGAAAAGATGAAGGAGACAGAGGAGGGGTGTAACCAGAAAG |
| | GTGCGTTCCTCGTAGAGAAGACATCTACCGAAGTCCAATGCAAAGG |
| | CGGGAATGTCTGCGTGCAAGCTCAGGATCGCTATTACAATTCCTCAT |
| | GCAGCAAGTGGGCATGTGTTCCCTGCAGAGTCCGATCGGTTCCTGG |
| | AGTAGGGGTACCTGGAGTGGGCAGGGTCATACCGGTCTCTGGACCT |
| | GCCAGGTGTCTTAGCCAGTCCCGAAACCTGCTGAAGACCACAGATG |
| | ACATGGTGAAGACGGCCAGAGAAAAGCTGAAACATTATTCCTGCAC |
| | TGCTGAAGACATCGATCATGAAGACATCACACGGGACCAAACCAGC |
| | ACATTGAAGACCTGTTTACCACTGGAACTACACAAGAACGAGAGTT |
| | GCCTGGCTACTAGAGAGACTTCTTCCACAACAAGAGGGAGCTGCCT |
| | GCCCCCACAGAAGACGTCTTTGATGATGACCCTGTGCCTTGGTAGC |
| | ATCTATGAGGACTTGAAGATGTACCAGACAGAGTTCCAGGCCATCA |
| | ACGCAGCACTTCAGAATCACAACCATCAGCAGATCATTCTAGACAA |
| | GGGCATGCTGGTGGCCATCGATGAGCTGATGCAGTCTCTGAATCAT |
| | AATGGCGAGACTCTGCGCCAGAAACCTCCTGTGGGAGAAGCAGACC |
| | CTTACAGAGTGAAAATGAAGCTCTGCATCCTGCTTCACGCCTTCAGC |
| | ACCCGCGTCGTGACCATCAACAGGGTGATGGGCTATCTGAGCTCCG |
| | CCGGCGGCGGCGGGTCCCATCCAGTTGGACTGCTGGCCCGCGGTGG |
| | AGGCGGAAGTAACACACTTGTGCTCTTTGGGGCAGGATTCGGCGCA |
| | GTAATAACAGTCGTCGTCATCGTTGTCATCATCAAATGCTTCTGTAA |
| | GCACAGAAGCTGTTTCAGAAGAAGAAATGAGGCAAGCAGAGAAAC |
| | AAACAACAGCCTTACCTTCGGGCCTGAAGAAGCATTAGCTGAACAG |
| | ACCGTCTTCCTTTAG |
| SEQ ID | ATGTGTCCTCAGAAGCTAACCATCTCCTGGTTTGCCATCGTTTTGCT |
| NO: 24 | GGTGTCTCCACTCATGGCCATGTGGGAGCTGGAGAAAGACGTTTAT |
| | GTTGTAGAGGTGGACTGGACTCCCGATGCCCCTGGAGAAACAGTGA |
| | ACCTCACCTGTGACACGCCTGAAGAAGATGACATCACCTGGACCTC |
| | AGACCAGAGACATGGAGTCATAGGCTCTGGAAAGACCCTGACCATC |
| | ACTGTCAAAGAGTTTCTAGATGCTGGCCAGTACACCTGCCACAAAG |
| | GAGGCGAGACTCTGAGCCACTCACATCTGCTGCTCCACAAGAAGGA |
| | AAATGGAATTTGGTCCACTGAAATTTTAAAAAATTTCAAAAACAAG |
| | ACTTTCCTGAAGTGTGAAGCACCAAATTACTCCGGACGGTTCACGT |
| | GCTCATGGCTGGTGCAAAGAAACATGGACTTGAAGTTCAACATCAA |
| | GAGCAGTAGCAGTTCCCCTGACTCTCGGGCAGTGACATGTGGAATG |
| | GCGTCTCTGTCTGCAGAGAAGGTCACACTGGACCAAAGGGACTATG |
| | AGAAGTATTCAGTGTCCTGCCAGGAGGATGTCACCTGCCCAACTGC |
| | CGAGGAGACCCTGCCCATTGAACTGGCGTTGGAAGCACGGCAGCAG |
| | AATAAATATGAGAACTACAGCACCAGCTTCTTCATCAGGGACATCA |
| | TCAAACCAGACCCGCCCAAGAACTTGCAGATGAAGCCTTTGAAGAA |
| | CTCACAGGTGGAGGTCAGCTGGGAGTACCCTGACTCCTGGAGCACT |
| | CCCCATTCCTACTTCTCCCTCAAGTTCTTTGTTCGAATCCAGCGCAA |
| | GAAAGAAAAGATGAAGGAGACAGAGGAGGGGTGTAACCAGAAAG |
| | GTGCGTTCCTCGTAGAGAAGACATCTACCGAAGTCCAATGCAAAGG |
| | CGGGAATGTCTGCGTGCAAGCTCAGGATCGCTATTACAATTCCTCAT |
| | GCAGCAAGTGGGCATGTGTTCCCTGCAGAGTCCGATCGGTTCCTGG |
| | AGTAGGGGTACCTGGAGTGGGCAGGGTCATACCGGTCTCTGGACCT |
| | GCCAGGTGTCTTAGCCAGTCCCGAAACCTGCTGAAGACCACAGATG |
| | ACATGGTGAAGACGGCCAGAGAAAAGCTGAAACATTATTCCTGCAC |
| | TGCTGAAGACATCGATCATGAAGACATCACACGGGACCAAACCAGC |
| | ACATTGAAGACCTGTTTACCACTGGAACTACACAAGAACGAGAGTT |
| | GCCTGGCTACTAGAGAGACTTCTTCCACAACAAGAGGGAGCTGCCT |
| | GCCCCCACAGAAGACGTCTTTGATGATGACCCTGTGCCTTGGTAGC |
| | ATCTATGAGGACTTGAAGATGTACCAGACAGAGTTCCAGGCCATCA |
| | ACGCAGCACTTCAGAATCACAACCATCAGCAGATCATTCTAGACAA |
| | GGGCATGCTGGTGGCCATCGATGAGCTGATGCAGTCTCTGAATCAT |
| | AATGGCGAGACTCTGCGCCAGAAACCTCCTGTGGGAGAAGCAGACC |
| | CTTACAGAGTGAAAATGAAGCTCTGCATCCTGCTTCACGCCTTCAGC |
| | ACCCGCGTCGTGACCATCAACAGGGTGATGGGCTATCTGAGCTCCG |
| | CCGGCGGCGGCGGGTCCCTTGCTCAGGCGGTGCGATCCAGCGGTGG |
| | AGGCGGAAGTAACACACTTGTGCTCTTTGGGGCAGGATTCGGCGCA |
| | GTAATAACAGTCGTCGTCATCGTTGTCATCATCAAATGCTTCTGTAA |
| | GCACAGAAGCTGTTTCAGAAGAAGAAATGAGGCAAGCAGAGAAAC |
| | AAACAACAGCCTTACCTTCGGGCCTGAAGAAGCATTAGCTGAACAG |
| | ACCGTCTTCCTTTAG |

1.-29. (canceled)

30. A membrane-cleavable chimeric protein, oriented from N-terminal to C-terminal, having the formula: 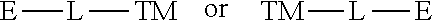
wherein:
a) E comprises an effector molecule;
b) L is a first linker comprising a protease cleavage site and an amino acid sequence selected from the group consisting of: GGGGSISSGLLSGRSDNHGGGGS (SEQ ID NO: 3); GGGGSVPLSLYSGGGISSGLLSGRSDNHGGGGS (SEQ ID NO: 4); GGGGSHPVGLLARGGGHPVGLLARGGSGRSAGGSGRSAGGGGS (SEQ ID NO: 5); GGGGSHPVGLLARGGGGS (SEQ ID NO: 6); and GGGGSLAQAVRSSGGGGS (SEQ ID NO: 7), wherein L is a cleavable linker;
c) TM comprises a cell transmembrane protein; and
d) E-L-TM or TM-L-E is configured to be expressed as a single polypeptide.

31. The membrane-cleavable chimeric protein of claim 30, wherein said membrane-cleavable chimeric protein further comprises a signal peptide at the N-terminus.

32. The membrane-cleavable chimeric protein of claim 30, wherein the effector molecule comprises a cytokine or a functional fragment thereof.

33. The membrane-cleavable chimeric protein of claim 32, wherein the cytokine is selected from the group consisting of: IL-1-beta, IL-2, IL-4, IL-6, IL-7, IL-10, IL-12, IL-15, IL-17A, IL-18, IL-21, IL-22, Type I interferons, Interferon-gamma, and TNF-alpha.

34. The membrane-cleavable chimeric protein of claim 33, wherein the cytokine is IL-12.

35. The membrane-cleavable chimeric protein of claim 30, wherein the effector molecule comprises p40 and p35 subunits of IL-12 linked by a second linker.

36. The membrane-cleavable chimeric protein of claim 30, wherein the transmembrane domain is derived from the group consisting of: PDGFR-beta, CD8, CD28, CD3 zeta-chain, CD4, 4-1BB, 0X40, ICOS, CTLA-4, PD-1, LAG-3, 2B4, LNGFR, NKG2D, EpoR, TNFR2, B7-1, and BTLA.

37. The membrane-cleavable chimeric protein of claim 30, wherein the transmembrane domain is derived from B7-1.

38. The membrane-cleavable chimeric protein of claim 30, wherein the transmembrane domain is linked to a cytoplasmic domain.

39. The membrane-cleavable chimeric protein of claim 38, wherein the cytoplasmic domain is derived from the group consisting of: PDGFR-beta, CD8, CD28, CD3 zeta-chain, CD4, 4-1BB, 0X40, ICOS, CTLA-4, PD-1, LAG-3, 2B4, LNGFR, NKG2D, EpoR, TNFR2, B7-1, and BTLA.

40. The membrane-cleavable chimeric protein of claim 39, wherein the cytoplasmic domain is derived from B7-1.

41. The membrane-cleavable chimeric protein of claim 30, wherein the effector molecule is cleaved from the chimeric protein upon cleavage of the cleavable linker L.

42. The membrane-cleavable chimeric protein of claim 30, wherein the cleavable linker L is preferentially cleaved in cancer cells.

43. The membrane-cleavable chimeric protein of claim 30, wherein
a) when expressed in a cell, the effector molecule is tethered to a cell membrane of the cell; and/or
b) when expressed in a cell expressing a protease capable of cleaving the protease cleavage site, the effector molecule is released from the cell membrane; and/or
c) wherein the protease expressed on the cell membrane is endogenous to the cell.

44. A nucleic acid molecule coding for the membrane-cleavable chimeric protein of claim 30.

45. A nucleic acid molecule comprising an expression cassette wherein the expression cassette comprises a promoter and an exogenous polynucleotide sequence encoding the membrane-cleavable chimeric protein of claim 30.

46. The nucleic acid molecule of claim 45, wherein the promoter is selected from the group consisting of a constitutive promoter, an inducible promoter, a tissue-specific promoter, and a synthetic promoter.

47. An expression vector comprising the nucleic acid molecule of claim 44.

48. The expression vector of claim 47, wherein the vector is a viral vector.

49. A cell comprising:
a) the membrane-cleavable chimeric protein of claim 30;
b) a nucleic acid encoding the membrane-cleavable chimeric protein; or
c) an expression vector comprising the nucleic acid molecule.

50. The cell of claim 49, wherein the cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell.

51. The cell of claim 49, wherein the cell further comprises a protease capable of cleaving the protease cleavage site.

52. The cell of claim 51, wherein the protease is an endogenous protease.

53. The cell of claim 49, wherein the membrane-cleavable chimeric protein is expressed in the cell and the effector molecule is tethered to a cell membrane of the cell.

54. The cell of claim 53, wherein the cell expresses a protease that cleaves the protease cleavage site and releases the effector molecule from the cell membrane.

55. A pharmaceutical composition comprising:
a) the membrane-cleavable chimeric protein of claim 30;
b) a nucleic acid encoding the membrane-cleavable chimeric protein;
c) an expression vector comprising the nucleic acid molecule; or
d) a cell comprising the membrane-cleavable chimeric protein, the nucleic acid or the expression vector,
and a pharmaceutically acceptable excipient.

56. The pharmaceutical composition of claim 55, wherein the pharmaceutical composition is a lipid nanoparticle pharmaceutical composition.

57. A method of treating a disease in a subject in need thereof, the method comprising administering a therapeutically effective amount of the pharmaceutical composition of claim 55.

58. The method of claim 57, wherein the disease is cancer.


SEQUENCE LISTING (24 sequences)

SEQ ID NO: 1 - protein, 15 aa, synthetic construct
GGGGSGGGGSGGGGS

SEQ ID NO: 2 - protein, 19 aa, synthetic construct
GGGGSGGGGSGGGGSGGGS

SEQ ID NO: 3 - protein, 23 aa, synthetic construct
GGGGSISSGLLSGRSDNHGGGGS

SEQ ID NO: 4 - protein, 33 aa, synthetic construct
GGGGSVPLSLYSGGGISSGLLSGRSDNHGGGGS

SEQ ID NO: 5 - protein, 43 aa, synthetic construct
GGGGSHPVGLLARGGGHPVGLLARGGSGRSAGGSGRSAGGGGS

SEQ ID NO: 6 - protein, 18 aa, synthetic construct
GGGGSHPVGLLARGGGGS

SEQ ID NO: 7 - protein, 18 aa, synthetic construct
GGGGSLAQAVRSSGGGGS

SEQ ID NO: 8 - protein, 13 aa, synthetic construct
ISSGLLSGRSDNH

SEQ ID NO: 9 - protein, 23 aa, synthetic construct
VPLSLYSGGGISSGLLSGRSDNH

SEQ ID NO: 10 - protein, 8 aa, synthetic construct
HPVGLLAR

SEQ ID NO: 11 - protein, 5 aa, synthetic construct
SGRSA

SEQ ID NO: 12 - protein, 8 aa, synthetic construct
LAQAVRSS

SEQ ID NO: 13 - protein, 8 aa, synthetic construct
LSGRSDNH

SEQ ID NO: 14 - protein, 8 aa, synthetic construct
ISSGLLSS

SEQ ID NO: 15 - skipped

SEQ ID NO: 16 - protein, 8 aa, synthetic construct
VPLSLYSG

SEQ ID NO: 17 - DNA, 1617 bp, synthetic construct
  source 1..1617: mol_type = other DNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagagtccgatcggttcctggagtaggggtacctggagtgggcagggtcataccggtctctggacctgccaggtgtcttagccagtcccgaaacctgctgaagaccacagatgacatggtgaagacggccagagaaaagctgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctga

SEQ ID NO: 18 - DNA, 1848 bp, synthetic construct
  source 1..1848: mol_type = other DNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagagtccgatcggttcctggagtaggggtacctggagtgggcagggtcataccggtctctggacctgccaggtgtcttagccagtcccgaaacctgctgaagaccacagatgacatggtgaagacggccagagaaaagctgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccggggggggaggcagtggaggcggtggttcaggtggtggtgggtccaacacacttgtgctctttggggcaggattcggcgcagtaataacagtcgtcgtcatcgttgtcatcatcaaatgcttctgtaagcacagaagctgtttcagaagaagaaatgaggcaagcagagaaacaaacaacagccttaccttcgggcctgaagaagcattagctgaacagaccgtcttcctttag

SEQ ID NO: 19 - DNA, 1848 bp, synthetic construct; identical to SEQ ID NO: 18
  source 1..1848: mol_type = other DNA

SEQ ID NO: 20 - DNA, 1872 bp, synthetic construct
  source 1..1872: mol_type = other DNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagagtccgatcggttcctggagtaggggtacctggagtgggcagggtcataccggtctctggacctgccaggtgtcttagccagtcccgaaacctgctgaagaccacagatgacatggtgaagacggccagagaaaagctgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccggaggtggagggtccataagtagtggactgctttctggccgaagtgataatcacggcggcggcggttccaacacacttgtgctctttggggcaggattcggcgcagtaataacagtcgtcgtcatcgttgtcatcatcaaatgcttctgtaagcacagaagctgtttcagaagaagaaatgaggcaagcagagaaacaaacaacagccttaccttcgggcctgaagaagcattagctgaacagaccgtcttcctttag

SEQ ID NO: 21 - DNA, 1902 bp, synthetic construct
  source 1..1902: mol_type = other DNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagagtccgatcggttcctggagtaggggtacctggagtgggcagggtcataccggtctctggacctgccaggtgtcttagccagtcccgaaacctgctgaagaccacagatgacatggtgaagacggccagagaaaagctgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccggcggcggcgggtccgttccattgtccctttattccgggggtggtatatcatcaggactgctcagcggacgatctgacaatcacggtggaggcggaagtaacacacttgtgctctttggggcaggattcggcgcagtaataacagtcgtcgtcatcgttgtcatcatcaaatgcttctgtaagcacagaagctgtttcagaagaagaaatgaggcaagcagagaaacaaacaacagccttaccttcgggcctgaagaagcattagctgaacagaccgtcttcctttag

SEQ ID NO: 22 - DNA, 1932 bp, synthetic construct
  source 1..1932: mol_type = other DNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagagtccgatcggttcctggagtaggggtacctggagtgggcagggtcataccggtctctggacctgccaggtgtcttagccagtcccgaaacctgctgaagaccacagatgacatggtgaagacggccagagaaaagctgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccggcggcggcgggtcccatccagttggactgctggcccgcggtgggggccacccggtggggctgttggcaagagggggctcagggcgcagcgctggtggtagtggcagaagtgcgggtggaggcggaagtaacacacttgtgctctttggggcaggattcggcgcagtaataacagtcgtcgtcatcgttgtcatcatcaaatgcttctgtaagcacagaagctgtttcagaagaagaaatgaggcaagcagagaaacaaacaacagccttaccttcgggcctgaagaagcattagctgaacagaccgtcttcctttag

SEQ ID NO: 23 - DNA, 1857 bp, synthetic construct
  source 1..1857: mol_type = other DNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagagtccgatcggttcctggagtaggggtacctggagtgggcagggtcataccggtctctggacctgccaggtgtcttagccagtcccgaaacctgctgaagaccacagatgacatggtgaagacggccagagaaaagctgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccggcggcggcgggtcccatccagttggactgctggcccgcggtggaggcggaagtaacacacttgtgctctttggggcaggattcggcgcagtaataacagtcgtcgtcatcgttgtcatcatcaaatgcttctgtaagcacagaagctgtttcagaagaagaaatgaggcaagcagagaaacaaacaacagccttaccttcgggcctgaagaagcattagctgaacagaccgtcttcctttag

SEQ ID NO: 24 - DNA, 1857 bp, synthetic construct
  source 1..1857: mol_type = other DNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagagtccgatcggttcctggagtaggggtacctggagtgggcagggtcataccggtctctggacctgccaggtgtcttagccagtcccgaaacctgctgaagaccacagatgacatggtgaagacggccagagaaaagctgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccggcggcggcgggtcccttgctcaggcggtgcgatccagcggtggaggcggaagtaacacacttgtgctctttggggcaggattcggcgcagtaataacagtcgtcgtcatcgttgtcatcatcaaatgcttctgtaagcacagaagctgtttcagaagaagaaatgaggcaagcagagaaacaaacaacagccttaccttcgggcctgaagaagcattagctgaacagaccgtcttcctttag